(19) United States
(12) Patent Application Publication
(10) Pub. No.: US 2025/0134920 A1
(43) Pub. Date: May 1, 2025

(54) Treatment Of Myeloid Cell Dysfunction With Membrane Spanning 4-Domains A6A (MS4A6A) Inhibitors

(71) Applicant: Regeneron Pharmaceuticals, Inc., Tarrytown, NY (US)

(72) Inventors: Neelroop Parikshak, Tarrytown, NY (US); Manuel Allen Revez Ferreira, Tarrytown, NY (US); Adrian Campos, Tarrytown, NY (US); Joe Udeochu, Tarrytown, NY (US); Daria Zamolodchikov, Tarrytown, NY (US); Nicole Alessandri-Haber, Tarrytown, NY (US); Aris Baras, Tarrytown, NY (US)

(21) Appl. No.: 18/894,415

(22) Filed: Sep. 24, 2024

Related U.S. Application Data
(60) Provisional application No. 63/546,362, filed on Oct. 30, 2023.
(60) Provisional application No. 63/546,965, filed on Nov. 2, 2023.

(51) Int. Cl. A61K 31/713; C12N 15/113

(52) U.S. Cl. CPC A61K 31/713; C12N 15/113; C12N 2310/11; C12N 2310/122; C12N 2310/14

(57) ABSTRACT

The present disclosure generally relates to the treatment of subjects having myeloid cell dysfunction or at risk of developing myeloid cell dysfunction by administering a Membrane Spanning 4-Domains A6A (MS4A6A) inhibitor and/or a Membrane Spanning 4-Domains A64A (MS4A4A) inhibitor to the subject.

### FIELD

The present disclosure generally relates to the treatment of subjects having myeloid cell dysfunction or at risk of developing myeloid cell dysfunction, by administering a Membrane Spanning 4-Domains A6A (MS4A6A) inhibitor to the subject, and to methods of identifying subjects having an increased risk of developing myeloid cell dysfunction.

### BACKGROUND

Alzheimer's Disease (AD) is a neurodegenerative disorder characterized by progressive dementia, loss of cognitive abilities, and deposition of fibrillar amyloid proteins as intraneuronal neurofibrillary tangles, extracellular amyloid plaques and vascular amyloid deposits, and is an example of myeloid cell dysfunction. The major constituents of these plaques are neurotoxic amyloid-beta protein 40 and amyloid-beta protein 42, that are produced by the proteolysis of the transmembrane APP protein. The cytotoxic C-terminal fragments (CTFs) and the caspase-cleaved products, such as C31, are also implicated in neuronal death. AD can be classified as early-onset or late-onset. The signs and symptoms of the early-onset form appear between a person's thirties and mid-sixties, while the late-onset form appears during or after a person's mid-sixties. The early-onset form is much less common than the late-onset form, accounting for less than 10 percent of all cases of AD. Other common symptoms include agitation, restlessness, withdrawal, and loss of language skills. There are no treatments that can stop the progression of AD, but certain drugs can temporarily slow worsening of some symptoms.

Triggering Receptor Expressed On Myeloid Cells 2 (TREM2) is a microglial receptor implicated in enhancing survival, lipid metabolism, proliferation, and phagocytosis. TREM2 agonism has been identified as a promising strategy for augmenting microglia function in AD.

Membrane Spanning 4-Domains A6A (MS4A6A) is encoded by a 13 kb gene located at 11q12.2. MS4A6A is 248 amino acids long and is a 27 kDa cell membrane protein that may be involved in signal transduction as a component of a multimeric receptor complex in the regulation of calcium signaling. Members of this nascent membrane-spanning 4A protein family are characterized by common structural features and similar intron/exon splice boundaries and display unique expression patterns among hematopoietic cells and nonlymphoid tissues.

### SUMMARY

The present disclosure provides methods of treating a subject having myeloid cell dysfunction, or at risk of developing myeloid cell dysfunction, the methods comprising administering an administering an MS4A6A inhibitor or the combination of an MS4A6A inhibitor and an MS4A4A inhibitor to the subject.

The present disclosure also provides methods of treating a subject having myeloid cell dysfunction or at risk of developing myeloid cell dysfunction by administering a myeloid cell dysfunction agent, the methods comprising: determining or having determined whether the subject has an MS4A6A variant nucleic acid molecule, by: obtaining or having obtained a biological sample from the subject; and performing or having performed a sequence analysis on the biological sample to determine if the subject has a genotype comprising an MS4A6A variant nucleic acid molecule; and administering or continuing to administer the myeloid cell dysfunction agent in an amount that is the same as or less than a standard dosage amount, and/or administering an MS4A6A inhibitor or the combination of an MS4A6A inhibitor and an MS4A4A inhibitor to a subject that is MS4A6A reference; administering or continuing to administer the myeloid cell dysfunction agent in an amount that is the same as or less than a standard dosage amount, and/or administering an MS4A6A inhibitor or the combination of an MS4A6A inhibitor and an MS4A4A inhibitor to a subject that is heterozygous for the MS4A6A variant nucleic acid molecule; or administering or continuing to administer the myeloid cell dysfunction agent in a standard dosage amount to a subject that is homozygous for the MS4A6A variant nucleic acid molecule; wherein the presence the MS4A6A variant nucleic acid molecule indicates the subject has a decreased risk of developing myeloid cell dysfunction.

The present disclosure also provides methods of identifying a subject having an increased risk of developing myeloid cell dysfunction, the methods comprising: determining or having determined the presence or absence of an MS4A6A variant nucleic acid molecule in a biological sample obtained from the subject; wherein: when the subject is MS4A6A reference, then the subject has an increased risk of developing myeloid cell dysfunction; and when the subject is heterozygous or homozygous for the MS4A6A variant nucleic acid molecule, then the subject has a decreased risk of developing myeloid cell dysfunction.

The present disclosure also provides myeloid cell dysfunction agents for use in the treatment or prevention of myeloid cell dysfunction in a subject having an MS4A6A variant nucleic acid molecule.

The present disclosure also provides MS4A6A inhibitors for use in the treatment or prevention of myeloid cell dysfunction in a subject that is MS4A6A reference or is heterozygous for an MS4A6A variant nucleic acid molecule.

The present disclosure also provides the combination of an MS4A6A inhibitor and an MS4A4A inhibitor for use in the treatment or prevention of myeloid cell dysfunction in a subject that is MS4A6A reference or is heterozygous for an MS4A6A variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows variants in the MS4 locus independently associated with soluble TREM2 levels and their effect on risk of AD. The effect of each variant was estimated separately (i.e., estimates reflect marginal effects).

FIG. 2 shows variants in the MS4 locus independently associated with soluble TREM2 levels and their effect on risk of AD. The effect of each variant was estimated in a model containing all variants (i.e., estimates reflect joint effects).

FIG. 3 shows the predicted functional impact of variants in the MS4 locus that are independently associated with soluble TREM2 plasma levels.

FIG. 4 shows LoF variants in MS4A6A increase soluble TREM2 levels, the association of which is explained by: i) rare splice donor; ii) a rarer frameshift; and iii) other even rarer pLoFs.

FIG. 5 shows LoF variants in MS4A6A have association with lower risk of AD.

FIG. 6 shows that inhibition of MS4A6A may be protective for AD in individuals who are carriers of pathogenic alleles in MS4A6A such as alleles that lower soluble TREM2.

FIG. 7 shows siRNA knockdown of MS4A4A or MS4A6A and potentiates TREM2-dependent signaling in differentiated THP1 macrophages.

FIG. 8 shows decreased microglia clustering around Aβ plaques which trend towards decreased Aβ deposition in Ms4a6* triple KO mice suggesting altered microglia response to Aβ pathology.

FIG. 9 shows that Alzheimer's disease cases have lower levels of plasma soluble TREM2 compared to controls and that Alzheimer's disease risk (as measured by the odds of reporting Alzheimer's) decrease with higher plasma sTREM2 in a dose dependent manner.

FIG. 10 shows that genetic risk scores based on the variants and effect sizes shown in FIG. 2 are associated with higher levels of plasma sTREM2 and with decreased Alzheimer's disease risk (as measured by the odds of reporting Alzheimer's).

FIG. 11 shows that genetic risk scores based on the variants and effect sizes shown in FIG. 2 are associated with changes in the cortical thickness and volumes of specific brain regions. GRS for sTREM2 was associated with higher cortical thickness of the inferior temporal (right hemisphere). In turn, participants with Alzheimer's disease case definitions showed reduced cortical thickness of the inferior temporal (right hemisphere) brain region.

FIG. 12 shows that effect of MS4A6A putative loss of function (pLoF) on measured plasma proteins (p<0.01) is concordant with the MS4A6A Thr185Ser missense variant effect on those same proteins.

FIG. 13 shows Western blot to confirm MS4A6A protein knockdown (Panel A); increased shed TREM2 in MS4A6A knockout pool compared to WT (Panel B); quantification of abundance measured by JESS in Panel B (Panel C); and MS4A6A WB and sTREM2 Jess in two different MS4A6A knockout THP1 lines (Panel D).

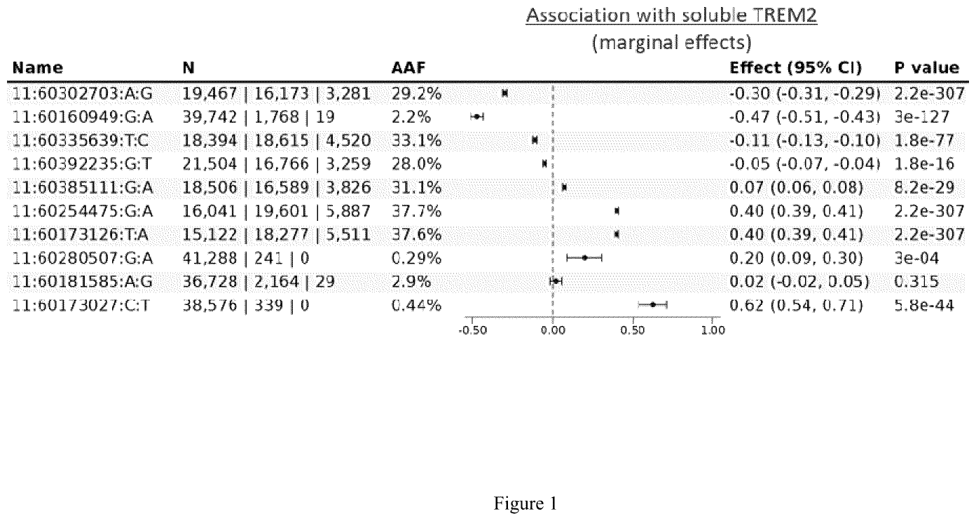

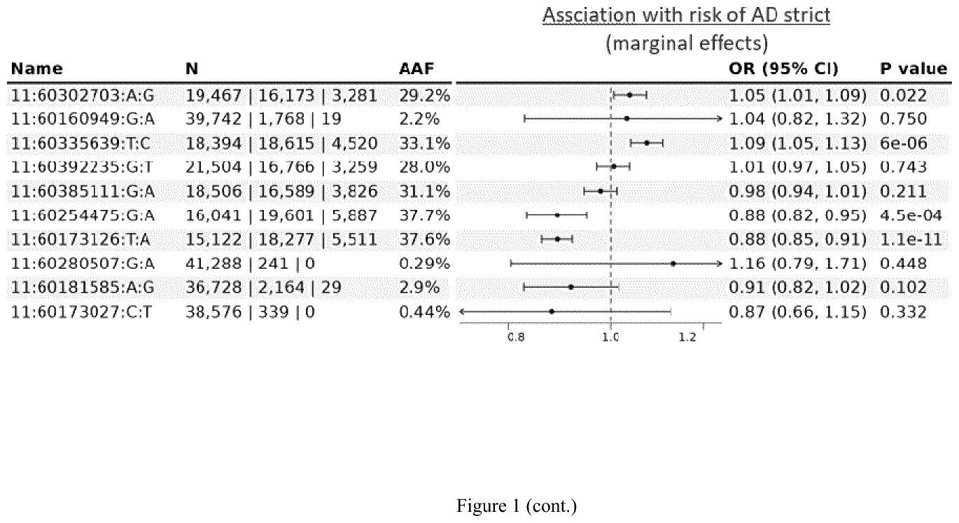

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician.

It has been observed in accordance with the present disclosure that individuals with a high genetic score for sTREM2 (top 20%), which corresponds to 28% higher sTREM2 in plasma, have 14% lower risk of AD (P=7×10⁻⁶). It has also been observed that common missense and rare LoF variants in MS4A6A substantially increased plasma sTREM2: i) heterozygotes for a rare splice donor (11:60173027:C:T) have 60% higher sTREM2 (P=2×10⁻¹⁰⁹); ii) heterozygotes for a rare frameshift (11:60179911:AT:A) have 40% higher STREM2 (P=5×10⁻⁵); and iii) homozygotes for a common missense (Thr185Ser) have 60% higher sTREM2 (P=4×10⁻¹⁴²) and 18% lower AD risk. Thus, rare and common MS4A6A variant nucleic acid molecules (whether these variants are homozygous or heterozygous in a particular subject) have been found to associate with a decreased risk of developing AD. It is believed that MS4A6A variant nucleic acid molecules have not been associated with myeloid cell dysfunction in humans. Therefore, subjects that are MS4A6A reference or heterozygous for an MS4A6A variant nucleic acid molecule may be treated with an MS4A6A inhibitor such that myeloid cell dysfunction is inhibited or prevented, the symptoms thereof are reduced or prevented, and/or development of symptoms is repressed or prevented. It is also believed that such subjects having myeloid cell dysfunction may further be treated with one or more myeloid cell dysfunction agents that treats or inhibits myeloid cell dysfunction. In addition, the present disclosure provides methods of leveraging the presence or absence of MS4A6A variant nucleic acid molecules in subjects to identify or stratify risk is such subjects of developing myeloid cell dysfunction, or to diagnose subjects as having an increased risk of developing myeloid cell dysfunction.

For purposes of the present disclosure, any particular subject, such as a human, can be categorized as having one of three MS4A6A genotypes: i) MS4A6A reference; ii) heterozygous for an MS4A6A variant nucleic acid molecule; or iii) homozygous for an MS4A6A variant nucleic acid molecule. A subject is MS4A6A reference when the subject does not have a copy of an MS4A6A variant nucleic acid molecule (i.e., homozygous for wild type MS4A6A that decrease sTREM2). A subject is heterozygous for an MS4A6A variant nucleic acid molecule when the subject has a single copy of an MS4A6A variant nucleic acid molecule. A subject is homozygous for an MS4A6A variant nucleic acid molecule when the subject has two copies of an MS4A6A variant nucleic acid molecule.

In any of the embodiments described herein, the MS4A6A variant nucleic acid molecule can be any nucleic acid molecule (such as, a genomic nucleic acid molecule, an mRNA molecule, or a cDNA molecule produced from an mRNA molecule) encoding an MS4A6A variant polypeptide having a partial loss-of-function, a complete loss-of-function, a predicted partial loss-of-function, or a predicted complete loss-of-function. A subject who has an MS4A6A polypeptide having a partial loss-of-function (or predicted partial loss-of-function) is hypomorphic for MS4A6A. In some embodiments, the MS4A6A variant nucleic acid molecule results in decreased or aberrant expression or activity of MS4A6A mRNA or polypeptide. In some embodiments, the MS4A6A variant nucleic acid molecule is associated with a reduced in vitro response to MS4A6A ligands compared with reference MS4A6A. In some embodiments, the MS4A6A variant nucleic acid molecule is a splice-site variant, a stop-gain variant, a start-loss variant, a stop-loss variant, a frameshift variant, an in-frame indel variant, or a variant that encodes a truncated MS4A6A variant polypeptide. In some embodiments, the MS4A6A variant nucleic acid molecule is a missense variant nucleic acid molecule. In some embodiments, the MS4A6A variant nucleic acid molecule comprises a single nucleotide polymorphism (SNP). In some embodiments, the MS4A6A variant nucleic acid molecule comprises a variation in a coding region. In some embodiments, the MS4A6A variant nucleic acid molecule does not comprise a variation in a non-coding region, except for a splice acceptor region (two bases before the start of any exon except the first). In some embodiments, the MS4A6A variant nucleic acid molecule results or is predicted to result in a premature truncation of an MS4A6A polypeptide compared to the reference MS4A6A. In some embodiments, the MS4A6A variant nucleic acid molecule is a variant that is predicted to be damaging to the protein function (and hence, in this case, protective to the human) by in vitro prediction algorithms such as Polyphen, SIFT, or similar algorithms. In some embodiments, the MS4A6A variant nucleic acid molecule is a variant that causes or is predicted to cause a nonsynonymous amino acid substitution in an MS4A6A nucleic acid molecule and whose allele frequency is less than 1/100 alleles in the population from which the subject is selected. In some embodiments, the MS4A6A variant nucleic acid molecule is any rare missense variant (allele frequency<0.1%; or 1 in 1,000 alleles), or any splice-site, stop-gain, start-loss, stop-loss, frameshift, or in-frame indel, or other frameshift MS4A6A variant.

In any of the embodiments described herein, the MS4A6A variant genomic nucleic acid molecule may include one or more variations at any of the positions of chromosome 11 (i.e., positions 60,172,015 to 60,184,666) using the nucleotide sequence of the MS4A6A reference genomic nucleic acid molecule in the GRCh38/hg38 human genome assembly (see, ENSG00000110077.15, ENST00000530839.5 annotated in the in the Ensembl database (URL: world wide web at “https://useast.ensembl.org/Homo_sapiens/Transcript/Summary?db=core;g=ENSG00000110077;r=11:60172015-60184666;t=ENST00000530839”)) as a reference sequence, including any isoform derived through alternative splicing or or post-translational processing. The sequences provided in these transcripts for the MS4A6A genomic nucleic acid molecule are only exemplary sequences. Other sequences for the MS4A6A genomic nucleic acid molecule are also possible.

In any of the embodiments described herein, the MS4A6A variant nucleic acid molecule may comprise any one or more of the following genetic variations in the genomic nucleic acid molecule (referring to the chromosome: positions set forth in the GRCh38/hg38 human genome assembly) in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs), as well as any other coding or non coding (i.e., intronic or 5′ UTR) genetic variations, or an mRNA molecule produced therefrom, or a cDNA molecule produced from the mRNA molecule. A list of MS4A6A variant nucleic acid molecules are shown in Table 1.

TABLE 1

| Variant | rsID |
|---|---|
| 11:60172159:T:A | rs759738457 |
| 11:60172160:A:C | rs1387404525 |
| 11:60172162:G:A | |
| 11:60172165:GTCAA:G | |
| 11:60172165:G:T | |
| 11:60172178:C:T | |
| 11:60172181:A:G | rs1310417040 |
| 11:60172183:C:T | rs144472637 |
| 11:60172183:C:G | |
| 11:60172184:C:T | |
| 11:60172186:C:T | |
| 11:60172189:T:A | |
| 11:60172192:T:C | rs368940475 |
| 11:60172193:G:T | |
| 11:60172193:G:A | |
| 11:60172195:G:A | rs753827016 |
| 11:60172196:T:G | |
| 11:60172196:T:C | rs757036088 |
| 11:60172197:C:T | |
| 11:60172198:AT:A | rs1308512030 |
| 11:60172198:A:T | |
| 11:60172202:T:C | rs1462202993 |
| 11:60172205:AG:A | |
| 11:60172207:G:C | |
| 11:60172207:GAC:G | |
| 11:60172208:A:G | |
| 11:60172211:T:C | rs765149120 |
| 11:60172213:C:A | rs750186088 |
| 11:60172213:C:T | rs750186088 |
| 11:60172222:C:T | rs1421281914 |
| 11:60172223:C:T | |
| 11:60172223:C:G | |
| 11:60172225:A:G | rs758006775 |
| 11:60172226:T:C | |
| 11:60172228:T:C | |
| 11:60172229:A:G | |
| 11:60172229:A:T | |
| 11:60172232:T:C | rs779592235 |
| 11:60172233:G:C | |
| 11:60172234:T:C | |
| 11:60172235:G:A | rs971378206 |
| 11:60172235:G:T | |
| 11:60172235:G:C | |
| 11:60172238:G:T | rs746487635 |
| 11:60172240:A:T | |
| 11:60172241:G:C | |
| 11:60173047:G:T | rs1304345958 |
| 11:60173048:C:T | |
| 11:60173049:C:T | |
| 11:60173056:C:T | rs762785825 |
| 11:60173057:A:G | |
| 11:60173057:A:T | |
| 11:60173059:C:G | |
| 11:60173059:C:T | rs146398167 |
| 11:60173060:G:A | rs371493203 |
| 11:60173060:G:T | rs371493203 |
| 11:60173061:C:T | |
| 11:60173062:A:G | |
| 11:60173062:A:T | rs139777263 |
| 11:60173065:A:C | |
| 11:60173066:C:A | rs1204184637 |
| 11:60173071:G:A | rs767302691 |
| 11:60173073:G:A | |
| 11:60173075:G:A | rs752214934 |
| 11:60173078:C:T | rs201213606 |
| 11:60173078:C:G | rs201213606 |
| 11:60173079:A:G | |
| 11:60173080:G:A | rs1248677682 |
| 11:60173081:C:G | |
| 11:60173083:A:G | |
| 11:60173084:G:A | |
| 11:60173086:C:G | |
| 11:60173086:C:A | |
| 11:60173086:C:T | |
| 11:60173090:A:G | rs1329476921 |
| 11:60173091:T:C | rs142963878 |
| 11:60173093:C:T | |
| 11:60173096:G:A | rs1218948624 |
| 11:60173100:A:C | |
| 11:60173101:G:A | |
| 11:60173102:T:C | rs747384914 |
| 11:60173106:A:C | |
| 11:60173107:A:G | |
| 11:60173109:C:T | rs1279558637 |
| 11:60173109:C:A | |
| 11:60173110:A:G | |
| 11:60173111:G:A | |
| 11:60175434:T:C | rs767934406 |
| 11:60175434:T:A | |
| 11:60175434:TG:T | |
| 11:60175436:G:C | |
| 11:60175437:T:C | |
| 11:60175439:T:C | |
| 11:60175443:G:A | rs1168091734 |
| 11:60175446:A:G | |
| 11:60175448:T:A | |
| 11:60175449:C:A | rs1371575123 |
| 11:60175450:A:C | rs1409554517 |
| 11:60175451:T:G | |
| 11:60175451:T:C | |
| 11:60175453:A:T | |
| 11:60175454:TA:T | |
| 11:60175454:T:C | |
| 11:60175458:AG:A | |
| 11:60175459:G:C | rs756232802 |
| 11:60175463:G:C | rs749297129 |
| 11:60175466:A:G | |
| 11:60175471:A:T | |
| 11:60175472:CT:C | rs1342857717 |
| 11:60175472:C:A | |
| 11:60175472:C:T | |
| 11:60175474:T:A | |
| 11:60175475:C:A | rs1197496974 |
| 11:60175475:C:G | |
| 11:60175475:C:T | |
| 11:60175478:G:A | rs952456975 |
| 11:60175478:G:C | |
| 11:60175479:T:A | |
| 11:60175481:G:C | rs757463480 |
| 11:60175481:G:T | |
| 11:60175482:G:T | |
| 11:60175483:T:C | |
| 11:60175484:A:T | |
| 11:60175484:A:G | |
| 11:60175485:T:C | rs368985494 |
| 11:60175487:T:C | |
| 11:60175488:T:C | rs979871328 |
| 11:60175488:T:G | |
| 11:60175562:A:T | |
| 11:60175563:C:A | |
| 11:60175563:C:T | rs758365547 |
| 11:60175568:G:A | rs1162315021 |
| 11:60175577:G:A | |
| 11:60175580:C:T | rs1057044360 |
| 11:60175581:T:C | rs1364454192 |
| 11:60175583:A:C | |
| 11:60175584:G:C | |
| 11:60175587:T:C | |
| 11:60175587:T:A | |
| 11:60175589:C:A | |
| 11:60175595:A:T | |
| 11:60175596:C:A | rs1314712223 |
| 11:60175599:G:C | |
| 11:60175601:C:G | |
| 11:60175603:G:C | |
| 11:60175603:G:T | |
| 11:60175604:C:T | rs1354468341 |
| 11:60175604:C:A | |
| 11:60175607:T:C | rs371652355 |
| 11:60175608:G:A | |
| 11:60175608:G:C | |
| 11:60175611:C:A | rs772651842 |
| 11:60175611:C:T | |
| 11:60175612:C:G | rs762471480 |
| 11:60175613:T:C | rs772428170 |
| 11:60178259:C:G | |
| 11:60178260:CA:C | |
| 11:60178260:C:G | |
| 11:60178261:A:G | |
| 11:60178264:AG:A | rs770185417 |
| 11:60178265:G:C | |
| 11:60178265:G:A | |
| 11:60178265:G:T | rs778777907 |
| 11:60178267:T:C | |
| 11:60178268:T:C | |
| 11:60178271:T:C | |
| 11:60178273:A:T | |
| 11:60178276:C:A | |
| 11:60178280:T:C | |
| 11:60179755:C:A | rs767723186 |
| 11:60179755:C:T | |
| 11:60179757:G:T | |
| 11:60179757:G:A | |
| 11:60179758:G:T | |
| 11:60179758:G:A | |
| 11:60179760:G:A | rs1170844145 |
| 11:60179760:G:T | |
| 11:60179761:G:T | |
| 11:60179764:A:T | rs761556260 |
| 11:60179765:G:T | |
| 11:60179765:G:C | |
| 11:60179766:T:G | |
| 11:60179767:C:T | |
| 11:60179767:C:A | |
| 11:60179770:A:C | |
| 11:60179772:CT:C | |
| 11:60179772:C:A | |
| 11:60179773:T:C | rs764925451 |
| 11:60179775:T:C | |
| 11:60179776:TG:T | |
| 11:60179776:T:G | rs1332347046 |
| 11:60179776:T:A | |
| 11:60179778:G:A | |
| 11:60179779:C:T | rs583791 |
| 11:60179781:T:C | rs757973221 |
| 11:60179781:T:A | |
| 11:60179782:C:A | |
| 11:60179791:C:T | |
| 11:60179791:C:G | |
| 11:60179793:C:T | rs751125562 |
| 11:60179793:C:G | rs751125562 |
| 11:60179794:T:A | rs756638678 |
| 11:60179796:T:C | |
| 11:60179797:G:T | |
| 11:60179799:C:T | rs1195449006 |
| 11:60179802:A:C | |
| 11:60179802:A:G | rs1248642173 |
| 11:60179803:T:A | |
| 11:60179805:C:G | |
| 11:60179806:TG:T | |
| 11:60179874:G:T | |
| 11:60179875:A:C | |
| 11:60179877:G:A | |
| 11:60179880:A:G | |
| 11:60179882:T:A | rs147611482 |
| 11:60179883:TGG:T | |
| 11:60179884:G:A | rs766149183 |
| 11:60179884:G:T | |
| 11:60179890:A:C | rs751072418 |
| 11:60179892:T:C | |
| 11:60179895:G:A | |
| 11:60179896:G:A | |
| 11:60179898:G:T | |
| 11:60179898:G:A | rs754574003 |
| 11:60179899:A:G | |
| 11:60179900:G:T | |
| 11:60179901:A:G | |
| 11:60179902:A:T | rs2305431 |
| 11:60179904:G:T | |
| 11:60179905:A:G | |
| 11:60179907:G:T | rs185080144 |
| 11:60179907:G:A | rs185080144 |
| 11:60179908:C:G | |
| 11:60179908:C:A | |
| 11:60179908:C:T | |
| 11:60179910:G:A | rs1327797719 |
| 11:60179910:G:C | |
| 11:60179911:AT:A | rs532381110 |
| 11:60179911:A:G | |
| 11:60179911:AT:A | rs532381110 |
| 11:60179911:A:T | |
| 11:60179912:T:TA | rs1472914432 |
| 11:60179913:G:A | rs1163688397 |
| 11:60179914:C:T | |
| 11:60179919:A:G | rs772244377 |
| 11:60179920:T:A | |
| 11:60179920:T:C | |
| 11:60179923:T:C | rs1384137664 |
| 11:60179925:C:G | rs142312942 |
| 11:60179925:C:T | |
| 11:60179926:C:T | rs1314610281 |
| 11:60181613:T:C | |
| 11:60181613:T:G | |
| 11:60181619:G:T | |
| 11:60181620:G:C | |
| 11:60181620:G:T | rs539896615 |
| 11:60181622:T:C | |
| 11:60181627:T:C | |
| 11:60181631:C:T | rs1309185191 |
| 11:60181632:C:G | |
| 11:60181632:CT:C | rs778432731 |
| 11:60181633:T:G | rs201119654 |
| 11:60181633:T:G | rs201119654 |
| 11:60181634:G:A | |
| 11:60181634:G:C | |
| 11:60181637:T:C | |
| 11:60181639:G:A | rs1192410819 |
| 11:60181639:G:C | |
| 11:60181640:T:C | |
| 11:60181640:T:G | |
| 11:60181643:G:C | |
| 11:60181646:C:T | rs377754262 |
| 11:60181648:G:A | rs200243893 |
| 11:60181649:G:A | |
| 11:60181657:G:T | rs778801218 |
| 11:60181657:G:A | |
| 11:60181659:T:G | rs548637266 |
| 11:60181660:T:C | |
| 11:60181661:G:A | rs1460571884 |
| 11:60181661:G:C | |
| 11:60181663:G:C | |
| 11:60181664:A:C | |
| 11:60181664:A:G | |
| 11:60181667:A:G | |
| 11:60181668:G:T | rs867646126 |
| 11:60181670:T:G | rs148195696 |
| 11:60181673:T:G | rs1301800030 |
| 11:60181677:A:C | |
| 11:60181679:T:A | |
| 11:60181679:T:C | |
| 11:60181681:G:C | rs768274871 |
| 11:60181682:AT:A | |
| 11:60181735:T:C | |
| 11:60181738:C:T | |
| 11:60181738:C:A | |
| 11:60182976:A:G | rs1336556682 |
| 11:60182977:C:T | |
| 11:60183134:A:G | |
| 11:60183134:AC:A | |
| 11:60183135:C:T | |
| 11:60183139:T:C | rs558416882 |
| 11:60183141:G:A | |
| 11:60183142:T:C | rs1241093634 |
| 11:60183145:A:G | |
| 11:60183145:A:T | |
| 11:60183147:T:G | |
| 11:60183147:T:A | |
| 11:60183152:AAATGTTC:A | |
| 11:60183157:T:C | |
| 11:60183160:C:G | |
| 11:60183162:G:A | |
| 11:60183162:G:T | rs759808356 |
| 11:60183163:C:T | |
| 11:60183163:C:A | |
| 11:60183165:C:T | |
| 11:60183170:T:A | |
| 11:60172165:G:A | |
| 11:60172166:T:G | |
| 11:60172167:C:A | rs1452539704 |
| 11:60172171:A:T | |
| 11:60172175:C:T | |
| 11:60172244:A:G | rs778400703 |
| 11:60172247:G:T | |
| 11:60172252:C:A | |
| 11:60172253:T:C | rs749867768 |
| 11:60172254:C:T | |
| 11:60172254:C:G | rs140130948 |
| 11:60172255:T:A | |
| 11:60173000:GT:G | |
| 11:60173005:G:A | |
| 11:60173006:T:C | rs779319959 |
| 11:60173006:T:G | |
| 11:60173009:A:G | rs1350669395 |
| 11:60173011:C:A | |
| 11:60173011:C:T | |
| 11:60173012:C:T | rs367715554 |
| 11:60173015:C:A | rs772374894 |
| 11:60173021:C:T | |
| 11:60173023:C:T | rs780314709 |
| 11:60173024:T:C | |
| 11:60173026:AC:A | rs755725980 |
| 11:60173027:C:T | rs138650483 |
| 11:60173027:C:T | rs138650483 |
| 11:60173030:C:A | |
| 11:60173032:G:A | rs768873281 |
| 11:60173032:G:T | |
| 11:60173033:G:A | |
| 11:60173034:G:T | |
| 11:60173038:T:A | |
| 11:60173039:C:T | |
| 11:60173040:A:T | rs1299290097 |
| 11:60173041:G:A | |
| 11:60173041:G:T | |
| 11:60173041:G:C | |
| 11:60173042:A:C | |
| 11:60173042:AGT:A | |
| 11:60173042:A:G | |
| 11:60173043:G:A | |
| 11:60173043:G:C | rs1396524651 |
| 11:60173044:T:G | rs769727174 |
| 11:60173044:TAA:T | rs1390343278 |
| 11:60173045:A:C | |
| 11:60173115:CAG:C | |
| 11:60173115:C:G | rs1453973750 |
| 11:60173115:CAGAG:C | |
| 11:60173116:A:G | |
| 11:60173117:G:A | rs781420488 |
| 11:60173119:G:A | rs900570842 |
| 11:60173120:A:G | rs1162983070 |
| 11:60173121:G:C | rs376890624 |
| 11:60173121:G:T | |
| 11:60173123:G:A | |
| 11:60173124:AGT:A | |
| 11:60173124:A:G | rs17852114 |
| 11:60173124:A:T | |
| 11:60173126:T:A | rs7232 |
| 11:60173126:T:C | |
| 11:60173126:T:A | rs7232 |
| 11:60173127:T:C | |
| 11:60173128:C:T | |
| 11:60173131:T:C | rs1469575125 |
| 11:60175399:T:TA | |
| 11:60175400:A:G | |
| 11:60175403:G:A | |
| 11:60175403:G:T | |
| 11:60175404:C:T | |
| 11:60175404:CCAGA:C | |
| 11:60175404:C:A | rs1440597159 |
| 11:60175407:G:T | |
| 11:60175409:C:T | rs763824515 |
| 11:60175409:C:A | |
| 11:60175416:TG:T | |
| 11:60175422:T:C | |
| 11:60175424:T:G | rs370469349 |
| 11:60175424:T:C | rs370469349 |
| 11:60175425:A:G | |
| 11:60175426:G:T | |
| 11:60175427:C:G | rs752106359 |
| 11:60175431:C:G | |
| 11:60175433:GT:G | rs747540319 |
| 11:60175433:G:C | rs530964700 |
| 11:60175433:G:T | |
| 11:60175433:G:A | rs530964700 |
| 11:60175490:T:C | rs1249952121 |
| 11:60175491:T:C | rs1453513108 |
| 11:60175494:T:C | |
| 11:60175496:T:C | |
| 11:60175500:A:T | rs772143334 |
| 11:60175503:C:A | rs775094011 |
| 11:60175503:C:T | |
| 11:60175508:T:G | rs570232547 |
| 11:60175509:G:A | rs1429565678 |
| 11:60175509:G:T | rs1429565678 |
| 11:60175511:A:C | |
| 11:60175511:A:T | rs761287516 |
| 11:60175517:G:T | rs780600163 |
| 11:60175520:G:A | |
| 11:60175521:G:A | |
| 11:60175521:G:C | |
| 11:60175522:A:T | rs759969028 |
| 11:60175526:A:T | |
| 11:60175527:A:C | |
| 11:60175529:G:A | |
| 11:60175532:G:A | rs1298273168 |
| 11:60175533:C:T | |
| 11:60175533:C:G | rs768024458 |
| 11:60175535:T:C | |
| 11:60175536:G:C | rs761142514 |
| 11:60175538:T:C | rs764202114 |
| 11:60175539:TGA:T | rs1241563511 |
| 11:60175541:A:T | |
| 11:60175542:C:T | |
| 11:60175544:G:A | |
| 11:60175544:G:C | |
| 11:60175545:A:G | |
| 11:60175550:A:G | rs1249229414 |
| 11:60175551:T:A | |
| 11:60175554:T:C | rs757412173 |
| 11:60175555:GA:G | |
| 11:60175557:A:G | rs571016362 |
| 11:60175559:C:T | |
| 11:60175560:C:A | rs750548616 |
| 11:60175561:C:CT | |
| 11:60175562:A:G | |
| 11:60178281:C:A | rs1484260549 |
| 11:60178282:T:C | |
| 11:60178283:C:T | rs1225866056 |
| 11:60178285:G:A | |
| 11:60178286:T:C | |
| 11:60178289:C:T | rs141358081 |
| 11:60178289:C:T | rs141358081 |
| 11:60178292:T:A | |
| 11:60178292:T:C | rs754885000 |
| 11:60178296:TAGAG:T | |
| 11:60178300:G:A | rs900700919 |
| 11:60178300:G:C | |
| 11:60178303:C:T | rs764908234 |
| 11:60178304:C:A | |
| 11:60178304:C:T | |
| 11:60178307:A:G | rs752449478 |
| 11:60178310:T:A | rs957454916 |
| 11:60178312:A:G | |
| 11:60178313:T:C | rs998170797 |
| 11:60178316:A:G | rs1430221064 |
| 11:60178317:C:T | |
| 11:60179730:TG:T | |
| 11:60179731:G:A | rs772207396 |
| 11:60179731:G:T | |
| 11:60179731:G:C | rs772207396 |
| 11:60179732:G:T | |
| 11:60179733:C:A | rs760549619 |
| 11:60179733:C:T | rs760549619 |
| 11:60179734:A:T | |
| 11:60179734:A:G | |
| 11:60179737:T:A | |
| 11:60179742:C:T | rs1014992546 |
| 11:60179743:A:T | |
| 11:60179743:A:C | |
| 11:60179746:G:T | |
| 11:60179748:A:G | rs753631587 |
| 11:60179750:G:C | rs1031827542 |
| 11:60179750:G:T | |
| 11:60179751:C:G | |
| 11:60179754:G:C | |
| 11:60179754:G:T | |
| 11:60179808:C:T | rs760790218 |
| 11:60179809:C:T | |
| 11:60179810:C:A | rs749633289 |
| 11:60179810:C:G | rs749633289 |
| 11:60179812:C:T | rs779192277 |
| 11:60179815:C:T | |
| 11:60179815:C:G | |
| 11:60179820:ACT:A | rs767248647 |
| 11:60179824:T:C | |
| 11:60179825:A:T | |
| 11:60179826:C:T | |
| 11:60179830:C:A | |
| 11:60179830:C:CA | rs755881843 |
| 11:60179830:CA:C | rs549670217 |
| 11:60179831:A:T | |
| 11:60179831:A:C | rs1484198501 |
| 11:60179832:A:G | rs201014833 |
| 11:60179833:A:C | |
| 11:60179835:A:C | |
| 11:60179835:A:G | |
| 11:60179835:A:T | |
| 11:60179836:A:T | rs375980883 |
| 11:60179837:GGGTCCTAT:G | |
| 11:60179838:G:C | rs1368769298 |
| 11:60179838:G:T | |
| 11:60179842:C:T | |
| 11:60179844:A:G | |
| 11:60179844:A:T | |
| 11:60179845:T:C | |
| 11:60179847:A:T | |
| 11:60179850:G:A | |
| 11:60179851:G:A | rs776485053 |
| 11:60179853:T:C | |
| 11:60179854:A:G | rs1257792980 |
| 11:60179854:A:T | |
| 11:60179856:G:A | rs1437204813 |
| 11:60179857:C:T | rs766394874 |
| 11:60179857:C:A | |
| 11:60179859:G:T | |
| 11:60179862:T:A | |
| 11:60179871:G:A | |
| 11:30179929:A:C | |
| 11:60179930:G:C | |
| 11:60179931:C:T | rs1342553562 |
| 11:60179932:T:G | rs529814098 |
| 11:60179937:A:G | rs1332790321 |
| 11:60179940:A:G | rs1210642908 |
| 11:60179942:C:T | |
| 11:60179944:T:C | rs776622879 |
| 11:60179949:C:A | |
| 11:60179952:A:G | |
| 11:60179955:A:G | rs747966380 |
| 11:60179958:T:G | |
| 11:60179958:TG:T | |
| 11:60179959:GGA:G | |
| 11:60179959:G:A | rs769781965 |
| 11:60179960:G:C | rs1268711509 |
| 11:60179962:T:G | |
| 11:60179965:T:C | |
| 11:60181580:C:T | rs767219700 |
| 11:60181582:C:G | |
| 11:60181585:A:G | rs61742546 |
| 11:60181585:A:G | rs61742546 |
| 11:60181586:T:C | |
| 11:60181588:A:G | |
| 11:60181589:C:T | rs563878900 |
| 11:60181590:T:A | |
| 11:60181592:T:A | |
| 11:60181594:A:G | |
| 11:60181598:C:G | |
| 11:60181598:C:A | |
| 11:60181601:C:T | rs199757938 |
| 11:60181601:C:A | |
| 11:60181601:C:T | rs199757938 |
| 11:60181602:G:C | rs113133013 |
| 11:60181604:G:A | rs1304115347 |
| 11:60181604:G:T | rs1304115347 |
| 11:60181606:A:G | rs1224944219 |
| 11:60181607:G:T | |
| 11:60181609:T:C | |
| 11:60181609:T:G | |
| 11:60181610:G:A | |
| 11:60181685:G:C | rs531174327 |
| 11:60181685:G:A | rs531174327 |
| 11:60181687:A:G | rs779374671 |
| 11:60181688:G:A | rs1205561270 |
| 11:60181690:A:G | |
| 11:60181690:A:T | |
| 11:60181691:C:T | rs766897573 |
| 11:60181691:C:G | |
| 11:60181692:T:C | |
| 11:60181693:A:G | rs751884736 |
| 11:60181694:T:C | rs759987804 |
| 11:60181695:G:C | |
| 11:60181696:A:T | rs748679570 |
| 11:60181697:T:C | rs141125435 |
| 11:60181697:T:A | |
| 11:60181699:G:A | rs1249771324 |
| 11:60181699:G:T | rs1249771324 |
| 11:60181700:T:G | |
| 11:60181701:C:G | rs1481627179 |
| 11:60181702:T:C | rs1475641565 |
| 11:60181703:C:A | |
| 11:60181705:T:C | |
| 11:60181706:T:C | rs1185691229 |
| 11:60181706:T:A | |
| 11:60181708:G:T | rs1419146147 |
| 11:60181709:G:A | |
| 11:60181711:A:C | |
| 11:60181712:C:G | |
| 11:60181712:C:T | |
| 11:60181714:G:A | |
| 11:60181714:G:T | |
| 11:60181717:T:C | |
| 11:60181723:G:A | rs1217897913 |
| 11:60181724:T:C | |
| 11:60181724:T:A | rs1426954303 |
| 11:60181726:A:G | rs200331262 |
| 11:60181727:T:A | |
| 11:60181729:A:T | |
| 11:60181730:T:C | rs1395324954 |
| 11:60181732:G:A | |
| 11:60181732:G:C | |
| 11:60183173:T:A | |
| 11:60183177:T:C | |
| 11:60183178:A:G | rs1021576148 |
| 11:60183180:A:T | |
| 11:60183181:C:T | |
| 11:60183182:T:A | |
| 11:60183183:T:C | |
| 11:60183184:C:A | |
| 11:60183184:C:G | |
| 11:60183188:C:A | rs1223157029 |
| 11:60183192:T:G | |
| 11:60183193:T:G | |
| 11:60183195:A:T | |
| 11:60183195:A:G | rs966975485 |
| 11:60183196:G:C | |
| 11:60183197:TA:T | |
| 11:60183198:A:T | rs1313766599 |
| 11:60183202:C:A | |
| 11:60183202:C:T | rs911420080 |
| 11:60183203:C:T | rs1001548250 |
| 11:60183203:C:G | |
| 11:60183204:A:G | |
| 11:60183205:T:C | |

In some embodiments, the MS4A6A variant nucleic acid molecule does not comprise any one or more of the following genetic variations in the genomic nucleic acid molecule (referring to the chromosome: positions set forth in the GRCh38/hg38 human genome assembly): 11:60173126:T:A (Thr185Ser), 11:60180901:C:G, or 11:60178272:T:C, or an mRNA molecule produced therefrom, or a cDNA molecule produced from the mRNA molecule.

For subjects that are genotyped or determined to be MS4A6A reference, such subjects have an increased risk of developing myeloid cell dysfunction. For subjects that are genotyped or determined to be either MS4A6A reference or heterozygous for an MS4A6A variant nucleic acid molecule, such subjects can be treated with an MS4A6A inhibitor.

In any of the embodiments described herein, the subject in whom myeloid cell dysfunction is prevented by administering an MS4A6A inhibitor may be anyone at risk for developing myeloid cell dysfunction including, but not limited to, subjects with a genetic predisposition for developing myeloid cell dysfunction. In any of the embodiments described herein, the methods can be used to improve myeloid cell dysfunction.

In any of the embodiments described herein, the MS4A6A predicted loss-of-function polypeptide can be any MS4A6A polypeptide having a partial loss-of-function, a complete loss-of-function, a predicted partial loss-of-function, or a predicted complete loss-of-function.

Any one or more (i.e., any combination) of the MS4A6A variant nucleic acid molecules described herein can be used within any of the methods described herein to determine whether a subject has an increased or decreased risk of developing myeloid cell dysfunction. The combinations of particular variants can form a mask used for statistical analysis of the particular correlation of MS4A6A and an increased or decreased risk of developing myeloid cell dysfunction. In some embodiments, the mask used for statistical analysis of the particular correlation of MS4A6A and an increased or decreased risk of developing myeloid cell dysfunction can exclude any one or more of these MS4A6A variant nucleic acid molecules described herein.

In any of the embodiments described herein, the subject can have myeloid cell dysfunction. In any of the embodiments described herein, the subject can be at risk of developing myeloid cell dysfunction. In any of the embodiments described herein, the myeloid cell can be microglial cells or macrophage cells, such as those associated with the central nervous system. In any of the embodiments described herein, the myeloid cell dysfunction is Alzheimer's disease (AD).

In any of the embodiments described herein, the methods can be used to treat a complication or co-morbidity of myeloid cell dysfunction or reduce the risk of developing the same.

In any of the embodiments described herein, the subject may have a TREM2 variant nucleic acid molecule that results in an increased risk of AD. Such TREM2 variant nucleic acid molecules may result in the decreased expression and/or activity of TREM2. Such TREM2 variant nucleic acid molecules may comprise any one or more of the following genetic variations in the genomic nucleic acid molecule (referring to the chromosome: positions set forth in the GRCh38/hg38 human genome assembly; see, ENSG00000095970.17, ENST00000373113.8 annotated in the in the Ensembl database; world wide web at “http://useast.ensembl.org/Homo_sapiens/Gene/Summary? g=ENSG00000095970;r=6:41158506-41163186;transcript=ENST00000373113.8” as a reference sequence): 6:41161514:C:T, 6:41161469:C:T, 6:41161557:G:A (Gln33*), 6:41161471:CTG:C (Arg62fs), 6:41161340:GC:G (Ala105fs), or 6:41161395:C:T (Asp87Asn), or an mRNA molecule produced therefrom, or a cDNA molecule produced from the mRNA molecule. A list of TREM2 variant nucleic acid molecules are shown in Table 2.

TABLE 2

| Variant | rsID |
|---|---|
| 6:41158607:C:G | |
| 6:41158607:C:T | rs1232354245 |
| 6:41158608:C:T | |
| 6:41158608:C:A | rs768583708 |
| 6:41158611:TCC:T | |
| 6:41158614:C:T | |
| 6:41158616:G:A | rs761884997 |
| 6:41158616:G:T | |
| 6:41158617:C:T | |
| 6:41158617:C:A | rs767767228 |
| 6:41158618:T:G | |
| 6:41158620:C:T | rs1484828242 |
| 6:41158622:A:G | |
| 6:41158625:G:A | rs560988938 |
| 6:41158629:AG:A | |
| 6:41158631:G:A | |
| 6:41158639:GT:G | rs749135711 |
| 6:41158640:T:G | |
| 6:41158641:G:A | |
| 6:41158643:T:A | |
| 6:41158644:C:T | |
| 6:41158646:G:A | |
| 6:41158647:G:A | rs766466972 |
| 6:41158649:A:G | |
| 6:41158652:G:C | |
| 6:41158653:T:C | rs753777378 |
| 6:41158655:G:A | |
| 6:41158701:G:A | rs1005368749 |
| 6:41158702:T:C | |
| 6:41158703:A:G | |
| 6:41158704:T:G | |
| 6:41158706:C:T | rs748257648 |
| 6:41158707:A:G | |
| 6:41158709:G:T | |
| 6:41158715:C:CT | |
| 6:41158716:T:A | rs200820365 |
| 6:41158718:G:C | |
| 6:41158718:G:A | rs766236034 |
| 6:41158719:T:A | rs776713120 |
| 6:41158719:TC:T | |
| 6:41158721:C:T | |
| 6:41158721:C:G | rs376505321 |
| 6:41158722:C:CTA | |
| 6:41158725:G:T | |
| 6:41158725:G:A | rs752213350 |
| 6:41158725:G:C | rs752213350 |
| 6:41158727:G:A | |
| 6:41158727:G:T | |
| 6:41158728:G:A | |
| 6:41158734:C:T | rs75272959 |
| 6:41158736:C:T | |
| 6:41158739:G:T | |
| 6:41158742:C:T | |
| 6:41158745:T:C | rs763760706 |
| 6:41158746:T:C | |
| 6:41158747:C:G | |
| 6:41158748:C:T | |
| 6:41158749:TC:T | rs1186372129 |
| 6:41158749:T:A | rs534753905 |
| 6:41158751:C:G | |
| 6:41158751:C:T | |
| 6:41158755:C:G | |
| 6:41158758:CT:C | |
| 6:41158759:T:A | |
| 6:41158766:A:C | rs1017615278 |
| 6:41158767:C:T | rs199795809 |
| 6:41158768:G:A | rs368921728 |
| 6:41158769:T:G | |
| 6:41158931:A:T | rs745696322 |
| 6:41158934:T:C | rs1179905275 |
| 6:41158935:GTCCCT:G | |
| 6:41158937:C:T | |
| 6:41158938:C:T | rs769593356 |
| 6:41158942:G:C | |
| 6:41158944:T:C | |
| 6:41158955:C:A | |
| 6:41158956:C:T | rs1228661369 |
| 6:41158959:G:A | rs768572417 |
| 6:41158960:C:T | |
| 6:41158963:C:T | rs1345120258 |
| 6:41158966:C:T | |
| 6:41158966:C:G | |
| 6:41158969:A:G | rs1279995205 |
| 6:41158970:G:C | |
| 6:41158971:A:T | |
| 6:41158973:G:A | rs1214159138 |
| 6:41158974:G:A | |
| 6:41158975:C:T | rs150277350 |
| 6:41158975:C:T | rs150277350 |
| 6:41158976:G:A | rs750046115 |
| 6:41158977:C:G | rs765569948 |
| 6:41158977:C:T | rs765569948 |
| 6:41158977:C:A | |
| 6:41158977:C:CT | |
| 6:41158979:G:A | |
| 6:41158980:G:A | |
| 6:41158980:G:T | rs541549712 |
| 6:41158986:A:G | |
| 6:41158989:A:G | |
| 6:41158990:T:C | rs1173193141 |
| 6:41158991:C:T | |
| 6:41158992:T:C | |
| 6:41158994:G:A | |
| 6:41158997:G:A | |
| 6:41158998:A:G | rs1425834660 |
| 6:41159004:A:T | |
| 6:41159004:A:C | |
| 6:41159005:T:C | |
| 6:41159006:G:T | rs778415288 |
| 6:41159042:G:C | rs769493472 |
| 6:41159042:G:A | rs769493472 |
| 6:41159043:G:A | |
| 6:41159044:G:T | |
| 6:41159045:G:C | |
| 6:41159045:G:T | rs1423781551 |
| 6:41159049:T:G | |
| 6:41159050:C:T | rs1274963337 |
| 6:41159052:C:T | rs779960537 |
| 6:41159053:C:T | rs267601028 |
| 6:41159056:C:T | |
| 6:41159057:C:A | |
| 6:41159063:G:C | rs371702633 |
| 6:41159068:T:G | rs768621570 |
| 6:41159071:G:A | rs1185237087 |
| 6:41159074:A:G | |
| 6:41159077:C:A | |
| 6:41159082:AG:A | |
| 6:41159083:G:A | rs761336507 |
| 6:41159088:A:T | |
| 6:41159089:T:TG | |
| 6:41159089:T:G | |
| 6:41159090:G:A | rs1457603816 |
| 6:41159092:G:A | rs1391899655 |
| 6:41159092:G:C | |
| 6:41159095:C:A | |
| 6:41159095:C:G | |
| 6:41159099:G:A | rs760316825 |
| 6:41159101:G:A | |
| 6:41159102:A:G | |
| 6:41159105:G:A | rs1288297122 |
| 6:41159107:C:A | |
| 6:41159107:C:T | rs538447052 |
| 6:41159107:C:G | |
| 6:41159790:A:G | rs386834144 |
| 6:41159792:C:T | |
| 6:41159793:T:A | rs539332069 |
| 6:41159795:G:A | rs1275430164 |
| 6:41159798:A:T | |
| 6:41159799:T:A | |
| 6:41159800:G:T | rs764489173 |
| 6:41159880:G:T | rs1257994892 |
| 6:41159881:G:T | rs139607688 |
| 6:41159882:T:C | rs762052858 |
| 6:41159883:C:T | |
| 6:41159884:T:A | |
| 6:41161265:G:A | rs201280312 |
| 6:41161266:C:T | rs759576705 |
| 6:41161267:CA:C | |
| 6:41161269:G:C | |
| 6:41161271:A:T | |
| 6:41161271:A:G | |
| 6:41161271:A:C | |
| 6:41161275:C:T | |
| 6:41161277:A:C | rs121908402 |
| 6:41161278:C:T | |
| 6:41161278:C:G | |
| 6:41161280:A:G | |
| 6:41161284:C:T | |
| 6:41161285:CT:C | rs1299091971 |
| 6:41161289:C:T | |
| 6:41161290:T:A | |
| 6:41161292:A:C | |
| 6:41161295:G:T | |
| 6:41161298:T:C | |
| 6:41161299:C:T | |
| 6:41161301:G:A | |
| 6:41161301:G:T | |
| 6:41161302:C:G | |
| 6:41161305:C:T | |
| 6:41161307:C:T | |
| 6:41161308:T:A | |
| 6:41161310:C:T | rs751357650 |
| 6:41161312:A:T | rs781244560 |
| 6:41161317:G:A | |
| 6:41161322:T:C | |
| 6:41161323:G:T | rs755641572 |
| 6:41161326:A:G | |
| 6:41161327:C:G | |
| 6:41161327:C:A | |
| 6:41161331:T:C | rs779888024 |
| 6:41161335:G:A | |
| 6:41161401:T:A | |
| 6:41161404:TG:T | rs773821396 |
| 6:41161404:TG:T | rs773821396 |
| 6:41161406:G:T | |
| 6:41161407:C:T | rs764373520 |
| 6:41161409:G:A | |
| 6:41161410:T:A | rs1425755566 |
| 6:41161412:C:G | |
| 6:41161412:C:T | rs1432256574 |
| 6:41161420:C:T | |
| 6:41161421:C:T | rs104893998 |
| 6:41161424:C:T | |
| 6:41161424:C:A | |
| 6:41161427:C:T | rs750350177 |
| 6:41161430:A:T | rs755692913 |
| 6:41161431:G:T | |
| 6:41161433:A:T | |
| 6:41161434:A:G | |
| 6:41161436:G:A | |
| 6:41161442:A:C | |
| 6:41161442:A:G | |
| 6:41161443:G:GCCACAAGT | |
| 6:41161450:G:C | rs753372932 |
| 6:41161451:T:A | |
| 6:41161451:T:C | rs1018859599 |
| 6:41161453:G:T | rs1229671015 |
| 6:41161454:TG:T | rs1294860969 |
| 6:41161455:G:T | |
| 6:41161455:G:A | rs1404747261 |
| 6:41161457:G:A | rs201258663 |
| 6:41161457:G:A | rs201258663 |
| 6:41161461:T:C | rs747377941 |
| 6:41161464:C:G | |
| 6:41161469:C:T | rs143332484 |
| 6:41161469:C:G | |
| 6:41161469:C:A | rs143332484 |
| 6:41161469:C:T | rs143332484 |
| 6:41161470:G:A | rs201258314 |
| 6:41161472:T:A | |
| 6:41161473:G:A | |
| 6:41161474:G:T | |
| 6:41161533:T:G | |
| 6:41161537:G:C | rs200392967 |
| 6:41161539:C:T | rs764816591 |
| 6:41161541:TA:T | rs1433830484 |
| 6:41161541:T:C | rs797044603 |
| 6:41161541:T:A | |
| 6:41161542:A:G | rs937164872 |
| 6:41161544:G:A | |
| 6:41161544:G:C | |
| 6:41161548:A:G | |
| 6:41161550:G:A | |
| 6:41161554:C:T | |
| 6:41161554:C:A | |
| 6:41161556:T:G | |
| 6:41161556:T:C | |
| 6:41161557:G:A | rs104894002 |
| 6:41161557:G:T | |
| 6:41161560:G:C | |
| 6:41161562:G:A | rs746216516 |
| 6:41161562:G:T | rs746216516 |
| 6:41161564:C:G | |
| 6:41161571:G:C | |
| 6:41161571:G:A | rs2234252 |
| 6:41161571:G:A | rs2234252 |
| 6:41161575:C:G | |
| 6:41161575:C:A | rs768745050 |
| 6:41161575:C:T | rs768745050 |
| 6:41161577:C:G | rs748326594 |
| 6:41161578:C:T | |
| 6:41161580:T:C | rs772412391 |
| 6:41161581:G:A | rs1434813037 |
| 6:41161584:A:G | |
| 6:41161584:A:T | |
| 6:41161587:CTG:C | |
| 6:41161587:C:G | rs1294900634 |
| 6:41161587:C:T | |
| 6:41161589:G:T | rs760511786 |
| 6:41161589:G:A | rs760511786 |
| 6:41161590:T:A | rs776578102 |
| 6:41161590:T:G | |
| 6:41158656:T:C | rs754547724 |
| 6:41158657:C:A | rs530314472 |
| 6:41158658:T:G | |
| 6:41158659:C:T | |
| 6:41158659:C:A | |
| 6:41158659:C:G | rs752403953 |
| 6:41158660:T:G | |
| 6:41158662:G:A | rs758041103 |
| 6:41158663:C:A | |
| 6:41158667:A:G | rs1439075848 |
| 6:41158670:A:G | rs746283732 |
| 6:41158671:G:T | |
| 6:41158674:C:A | |
| 6:41158674:C:T | |
| 6:41158682:C:A | |
| 6:41158682:C:T | |
| 6:41158684:TG:T | |
| 6:41158685:G:A | rs947954226 |
| 6:41158685:G:T | |
| 6:41158686:G:T | |
| 6:41158688:G:A | |
| 6:41158690:C:T | |
| 6:41158690:C:A | rs749732493 |
| 6:41158691:C:T | rs2234258 |
| 6:41158694:G:C | |
| 6:41158694:G:A | rs532556414 |
| 6:41158697:G:T | |
| 6:41158697:G:A | |
| 6:41158772:C:G | |
| 6:41158773:T:C | |
| 6:41158777:A:G | |
| 6:41158778:G:T | |
| 6:41158779:C:T | rs779444937 |
| 6:41158779:C:G | |
| 6:41158780:C:A | |
| 6:41158780:C:T | rs748227970 |
| 6:41158871:A:G | |
| 6:41158871:A:C | rs28479469 |
| 6:41158872:C:T | rs1270474102 |
| 6:41158875:G:A | rs1205300439 |
| 6:41158876:G:A | rs1276123772 |
| 6:41158878:A:G | |
| 6:41158881:G:A | rs138355759 |
| 6:41158881:G:A | rs138355759 |
| 6:41158884:T:C | |
| 6:41158885:G:T | |
| 6:41158888:G:T | |
| 6:41158893:T:C | |
| 6:41158894:AC:A | rs1309876002 |
| 6:41158896:C:G | rs777941887 |
| 6:41158899:G:C | |
| 6:41158899:G:A | |
| 6:41158903:C:A | rs373137055 |
| 6:41158904:A:C | rs1161481912 |
| 6:41158905:T:C | |
| 6:41158906:G:A | |
| 6:41158911:C:T | |
| 6:41158912:A:G | |
| 6:41158914:T:C | |
| 6:41158917:A:G | rs2234256 |
| 6:41158917:A:G | rs2234256 |
| 6:41158920:T:C | |
| 6:41158922:A:G | rs1036861193 |
| 6:41158923:C:T | rs1333444417 |
| 6:41158925:G:T | |
| 6:41158927:G:A | |
| 6:41158929:G:A | |
| 6:41158931:A:G | |
| 6:41158931:A:C | |
| 6:41159006:G:C | |
| 6:41159008:A:G | |
| 6:41159009:G:C | rs558684978 |
| 6:41159011:C:T | |
| 6:41159011:C:G | |
| 6:41159012:C:T | rs1287376094 |
| 6:41159012:C:A | |
| 6:41159012:C:G | |
| 6:41159013:A:C | |
| 6:41159013:A:G | |
| 6:41159014:G:A | |
| 6:41159015:G:C | |
| 6:41159015:G:T | rs1435456359 |
| 6:41159016:A:C | rs757387879 |
| 6:41159016:A:G | |
| 6:41159017:G:T | |
| 6:41159017:G:A | rs1368290241 |
| 6:41159018:G:A | |
| 6:41159020:G:A | |
| 6:41159021:A:G | |
| 6:41159023:G:A | rs1306444285 |
| 6:41159024:G:C | |
| 6:41159024:G:A | |
| 6:41159025:A:G | |
| 6:41159025:A:T | |
| 6:41159026:TG:T | |
| 6:41159026:T:C | |
| 6:41159028:G:A | |
| 6:41159031:G:C | |
| 6:41159032:T:C | |
| 6:41159033:G:A | |
| 6:41159033:G:T | |
| 6:41159034:G:A | |
| 6:41159035:G:A | rs781302866 |
| 6:41159036:T:A | |
| 6:41159036:T:G | |
| 6:41159037:G:A | |
| 6:41159038:G:A | |
| 6:41159039:G:C | rs138788407 |
| 6:41159039:G:C | rs138788407 |
| 6:41159040:A:G | |
| 6:41159801:C:T | |
| 6:41159805:G:A | rs2234255 |
| 6:41159805:G:A | rs2234255 |
| 6:41159811:C:T | |
| 6:41159812:A:T | |
| 6:41159813:T:A | |
| 6:41159813:T:G | |
| 6:41159816:G:A | rs1187953302 |
| 6:41159817:C:T | |
| 6:41159819:T:C | |
| 6:41159823:C:T | rs79011726 |
| 6:41159823:C:T | rs79011726 |
| 6:41159826:A:G | |
| 6:41159828:C:T | rs867253281 |
| 6:41159829:T:C | rs750556369 |
| 6:41159835:A:G | |
| 6:41159837:TC:T | |
| 6:41159838:C:G | rs1430713564 |
| 6:41159838:C:T | |
| 6:41159841:C:A | rs766647311 |
| 6:41159841:C:T | rs766647311 |
| 6:41159844:G:C | |
| 6:41159844:G:A | |
| 6:41159845:G:C | |
| 6:41159848:C:A | |
| 6:41159848:C:G | |
| 6:41159850:A:G | |
| 6:41159852:A:G | |
| 6:41159855:T:A | |
| 6:41159858:C:T | |
| 6:41159859:C:T | |
| 6:41159864:TC:T | |
| 6:41159865:C:T | rs758428150 |
| 6:41159867:C:T | rs149622783 |
| 6:41159867:C:T | rs149622783 |
| 6:41159868:G:A | rs772641807 |
| 6:41159868:G:C | |
| 6:41159873:T:C | rs28939079 |
| 6:41159876:AG:A | |
| 6:41159879:G:A | |
| 6:41159880:G:A | |
| 6:41161337:C:A | |
| 6:41161338:C:A | |
| 6:41161340:GC:G | rs386834141 |
| 6:41161340:G:A | rs145080901 |
| 6:41161340:G:A | rs145080901 |
| 6:41161341:C:A | |
| 6:41161342:A:T | |
| 6:41161343:T:C | rs1409131974 |
| 6:41161343:T:A | |
| 6:41161345:A:T | rs372594330 |
| 6:41161349:G:A | |
| 6:41161350:G:A | |
| 6:41161350:G:T | rs1173985669 |
| 6:41161352:T:G | rs750686409 |
| 6:41161359:T:C | |
| 6:41161361:C:A | |
| 6:41161361:C:T | rs374248493 |
| 6:41161362:G:A | rs147564421 |
| 6:41161362:G:A | rs147564421 |
| 6:41161364:A:G | rs1311610930 |
| 6:41161367:G:T | rs2234253 |
| 6:41161367:G:A | rs2234253 |
| 6:41161367:G:T | rs2234253 |
| 6:41161371:T:A | |
| 6:41161371:TG:T | |
| 6:41161373:G:A | |
| 6:41161373:G:C | |
| 6:41161374:TGA:T | |
| 6:41161377:G:C | rs914179215 |
| 6:41161379:G:A | |
| 6:41161379:G:C | |
| 6:41161383:C:T | |
| 6:41161385:C:T | rs1271841898 |
| 6:41161386:C:G | |
| 6:41161389:G:C | |
| 6:41161391:G:A | rs1483052893 |
| 6:41161391:G:T | |
| 6:41161395:C:T | rs142232675 |
| 6:41161395:C:T | rs142232675 |
| 6:41161396:GT:G | rs767770945 |
| 6:41161400:G:A | rs368255898 |
| 6:41161476:A:G | |
| 6:41161477:TG:T | |
| 6:41161478:G:A | rs1247550853 |
| 6:41161478:G:T | |
| 6:41161479:G:C | |
| 6:41161479:G:A | |
| 6:41161481:C:T | rs886042808 |
| 6:41161481:C:G | rs886042808 |
| 6:41161482:C:T | |
| 6:41161487:T:A | |
| 6:41161493:A:T | rs954383723 |
| 6:41161497:G:A | |
| 6:41161499:C:T | rs374851046 |
| 6:41161500:G:T | |
| 6:41161500:G:A | rs749358844 |
| 6:41161501:G:T | |
| 6:41161502:C:T | rs369181900 |
| 6:41161505:C:T | rs549402254 |
| 6:41161508:G:A | rs1359529169 |
| 6:41161508:G:T | |
| 6:41161509:C:G | |
| 6:41161509:C:T | rs767357967 |
| 6:41161509:C:A | rs767357967 |
| 6:41161510:C:A | |
| 6:41161514:C:A | rs75932628 |
| 6:41161514:C:T | rs75932628 |
| 6:41161514:C:T | rs75932628 |
| 6:41161515:G:C | |
| 6:41161515:G:A | rs753325601 |
| 6:41161518:TC:T | |
| 6:41161521:C:A | rs1299147476 |
| 6:41161521:C:G | |
| 6:41161521:C:T | |
| 6:41161522:C:T | rs104894001 |
| 6:41161523:C:G | |
| 6:41161526:T:A | |
| 6:41161526:T:C | rs1332526817 |
| 6:41161527:G:T | |
| 6:41161527:G:A | |
| 6:41161532:A:G | rs1395807649 |
| 6:41161532:A:C | |
| 6:41161595:T:G | rs534417680 |
| 6:41161596:T:TG | |
| 6:41161598:T:C | |
| 6:41161599:G:T | rs764738032 |
| 6:41161599:G:A | rs764738032 |
| 6:41161605:C:G | |
| 6:41161605:C:T | |
| 6:41161607:G:A | rs777808487 |
| 6:41161608:A:G | rs1451130693 |
| 6:41161610:A:G | |
| 6:41161611:G:T | |
| 6:41161611:GCT:G | |
| 6:41161612:C:A | |
| 6:41161614:C:A | rs750837494 |
| 6:41163042:C:A | |
| 6:41163042:C:T | rs766712618 |
| 6:41163043:C:A | rs386834143 |
| 6:41163049:CAA:C | rs1473834050 |
| 6:41163053:GA:G | |
| 6:41163056:T:G | |
| 6:41163061:T:C | |
| 6:41163063:A:G | |
| 6:41163064:G:A | rs372634215 |
| 6:41163067:GC:G | |
| 6:41163069:C:T | rs146485698 |
| 6:41163069:C:A | |
| 6:41163069:C:T | rs146485698 |
| 6:41163070:G:A | rs374483382 |
| 6:41163070:G:C | |
| 6:41163072:A:C | |
| 6:41163073:G:A | |
| 6:41163075:G:T | |
| 6:41163076:G:T | rs141301187 |
| 6:41163076:G:A | |
| 6:41163076:G:T | rs141301187 |
| 6:41163077:C:A | rs1187834290 |
| 6:41163078:T:G | |
| 6:41163079:C:T | |
| 6:41163079:C:G | |
| 6:41163081:A:G | rs770644483 |

The present disclosure provides methods of treating a subject having myeloid cell dysfunction or at risk of developing myeloid cell dysfunction, the methods comprising administering an MS4A6A inhibitor alone or in combination with a Membrane Spanning 4-Domains A4A (MS4A4A) inhibitor to the subject.

In some embodiments, the MS4A6A inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an MS4A6A nucleic acid molecule. In some embodiments, the antisense RNA, siRNA, or shRNA hybridizes to a sequence within an MS4A6A genomic nucleic acid molecule or mRNA molecule and decreases expression of the MS4A6A polypeptide in a cell in the subject. In some embodiments, the MS4A6A inhibitor comprises an antisense molecule that hybridizes to an MS4A6A genomic nucleic acid molecule or mRNA molecule and decreases expression of the MS4A6A polypeptide in a cell in the subject. In some embodiments, the MS4A6A inhibitor comprises an siRNA that hybridizes to an MS4A6A genomic nucleic acid molecule or mRNA molecule and decreases expression of the MS4A6A polypeptide in a cell in the subject. In some embodiments, the MS4A6A inhibitor comprises an shRNA that hybridizes to an MS4A6A genomic nucleic acid molecule or mRNA molecule and decreases expression of the MS4A6A polypeptide in a cell in the subject.

In some embodiments, the MS4A4A inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an MS4A4A nucleic acid molecule. In some embodiments, the antisense RNA, siRNA, or shRNA hybridizes to a sequence within an MS4A4A genomic nucleic acid molecule or mRNA molecule and decreases expression of the MS4A4A polypeptide in a cell in the subject. In some embodiments, the MS4A4A inhibitor comprises an antisense molecule that hybridizes to an MS4A4A genomic nucleic acid molecule or mRNA molecule and decreases expression of the MS4A4A polypeptide in a cell in the subject. In some embodiments, the MS4A4A inhibitor comprises an siRNA that hybridizes to an MS4A4A genomic nucleic acid molecule or mRNA molecule and decreases expression of the MS4A4A polypeptide in a cell in the subject. In some embodiments, the MS4A4A inhibitor comprises an shRNA that hybridizes to an MS4A4A genomic nucleic acid molecule or mRNA molecule and decreases expression of the MS4A4A polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N₆-methyladenosine, inosine, and N₇-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/i2FN/*mN/i2FN/mN/i2FN/mN/i2FN/mN/i2FN/mN/i2FN/mN/i2FN/mN/İ2FN/mN/i2FN/mN*N*N
wherein: “N” is the base; “2F” is a 2′-F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 mg to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the MS4A6A and/or MS4A4A inhibitor comprises a nuclease agent that induces one or more nicks or double-strand breaks at a recognition sequence(s) or a DNA-binding protein that binds to a recognition sequence within an MS4A6A or MS4A4A genomic nucleic acid molecule. The recognition sequence can be located within a coding region of the MS4A6A gene or MS4A4A gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the MS4A6A gene or MS4A4A gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an MS4A6A and/or MS4A4A genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of MS4A6A and/or MS4A4A nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an MS4A6A or MS4A4A genomic nucleic acid molecule or it can be a nickase that creates a single-strand break in an MS4A6A or MS4A4A genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of MS4A6A and/or MS4A4A genomic nucleic acid molecules can be generated by contacting a cell with a Cas protein and one or more gRNAs that hybridize to one or more gRNA recognition sequences within a target genomic locus in the MS4A6A and/or MS4A4A genomic nucleic acid molecule. The gRNA recognition sequence can include or be proximate to the start codon of an MS4A6A and/or MS4A4A genomic nucleic acid molecule or the stop codon of an MS4A6A and/or MS4A4A genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an MS4A6A and/or MS4A4A genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-31, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an MS4A6A and/or MS4A4A genomic nucleic acid molecule. An exemplary gRNA is a gRNA effective to direct a Cas enzyme to bind to or cleave an MS4A6A and/or MS4A4A genomic nucleic acid molecule, wherein the gRNA comprises a DNA-targeting segment that hybridizes to a gRNA recognition sequence within the MS4A6A and/or MS4A4A genomic nucleic acid molecule. Exemplary gRNAs comprise a DNA-targeting segment that hybridizes to a gRNA recognition sequence present within an MS4A6A and/or MS4A4A genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the MS4A6A and/or MS4A4A genomic nucleic acid molecule. The Cas protein can cleave the nucleic acid molecule at a site within or outside of the nucleic acid sequence present in the MS4A6A and/or MS4A4A genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the MS4A6A and/or MS4A4A genomic nucleic acid molecule to which a DNA-targeting segment of a gRNA will bind.

Such methods can result, for example, in an MS4A6A and/or MS4A4A genomic nucleic acid molecule in which a region of the MS4A6A and/or MS4A4A genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the MS4A6A and/or MS4A4A genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In any of the methods of treatment or prevention described herein, the subject being treated may comprise an MS4A6A variant nucleic acid molecule. In some embodiments, the subject being treated is heterozygous for the MS4A6A variant nucleic acid molecule. In some embodiments, the subject being treated is homozygous for the MS4A6A variant nucleic acid molecule. In some embodiments, the subject being treated is MS4A6A reference. The MS4A6A variant nucleic acid molecule can be any of the MS4A6A variant nucleic acid molecules disclosed herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs), as well as any other coding or non coding (i.e., intronic or 5′ UTR) genetic variations, or an mRNA molecule produced therefrom, or a cDNA molecule produced from the mRNA molecule.

In some embodiments, the methods of treatment or prevention further comprise detecting the presence or absence of an MS4A6A variant nucleic acid molecule in a biological sample from the subject. In some embodiments, the MS4A6A variant nucleic acid molecule can be any of the MS4A6A variant nucleic acid molecules disclosed herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides methods of treating a subject with a myeloid cell dysfunction agent that treats or inhibits myeloid cell dysfunction, wherein the subject has myeloid cell dysfunction or is at risk of developing myeloid cell dysfunction. The methods comprise determining whether the subject has an MS4A6A variant nucleic acid molecule by obtaining or having obtained a biological sample from the subject, and performing or having performed a sequence analysis on the biological sample to determine if the subject has a genotype comprising the MS4A6A variant nucleic acid molecule. In embodiments where the subject is MS4A6A reference, the methods further comprise administering or continuing to administer the myeloid cell dysfunction agent in an amount that is the same as or less than a standard dosage amount to the subject, and/or administering an MS4A6A inhibitor or the combination of an MS4A6A inhibitor and an MS4A4A inhibitor to the subject. In embodiments where the subject is heterozygous for the MS4A6A variant nucleic acid molecule, the methods further comprise administering or continuing to administer the myeloid cell dysfunction agent in an amount that is the same as or less than a standard dosage amount to the subject, and/or administering an MS4A6A inhibitor or the combination of an MS4A6A inhibitor and an MS4A4A inhibitor to the subject. In embodiments where the subject is homozygous for the MS4A6A variant nucleic acid molecule, the methods further comprise administering or continuing to administer the myeloid cell dysfunction agent in a standard dosage amount to the subject. The presence of an MS4A6A variant nucleic acid molecule indicates the subject has a decreased risk of developing myeloid cell dysfunction. In some embodiments, the subject is MS4A6A reference. In some embodiments, the subject is heterozygous for an MS4A6A variant nucleic acid molecule. In some embodiments, the subject is homozygous for an MS4A6A variant nucleic acid molecule. In any of the embodiments described herein, the MS4A6A inhibitor is an example of a myeloid cell dysfunction agent. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

For subjects that are genotyped or determined to be either MS4A6A reference or heterozygous for an MS4A6A variant nucleic acid molecule, such subjects can be administered an MS4A6A inhibitor or the combination of an MS4A6A inhibitor and an MS4A4A inhibitor, as described herein.

Detecting the presence or absence of an MS4A6A variant nucleic acid molecule in a biological sample from a subject and/or determining whether a subject has an MS4A6A variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is MS4A6A reference, the subject is administered a myeloid cell dysfunction agent in an amount that is the same as or less than a standard dosage amount, and/or an MS4A6A inhibitor or the combination of an MS4A6A inhibitor and an MS4A4A inhibitor. In some embodiments, when the subject is heterozygous for an MS4A6A variant nucleic acid molecule, the subject is administered a myeloid cell dysfunction agent in an amount that is the same as or less than a standard dosage amount, and/or an MS4A6A inhibitor or the combination of an MS4A6A inhibitor and an MS4A4A inhibitor.

In some embodiments, the treatment or prevention methods comprise detecting the presence or absence of a decrease in the expression of an MS4A6A variant mRNA or polypeptide in a biological sample from the subject. In some embodiments, when the subject does not have a decrease in the expression of an MS4A6A variant mRNA or polypeptide, the subject is administered a myeloid cell dysfunction agent in an amount that is the same as or less than a standard dosage amount, and/or an MS4A6A inhibitor or the combination of an MS4A6A inhibitor and an MS4A4A inhibitor. In some embodiments, when the subject has a decrease in the expression of an MS4A6A variant mRNA or polypeptide, the subject is administered a myeloid cell dysfunction agent in a standard dosage amount.

The present disclosure also provides methods of treating a subject with a myeloid cell dysfunction agent that treats or inhibits myeloid cell dysfunction, wherein the subject has myeloid cell dysfunction or is at risk of developing myeloid cell dysfunction. The methods comprise determining whether the subject has a decrease in the expression of an MS4A6A variant mRNA or polypeptide by obtaining or having obtained a biological sample from the subject, and performing or having performed an assay on the biological sample to determine if the subject a decrease in the expression of an MS4A6A variant mRNA or polypeptide. In embodiments where the subject does not have a decrease in the expression of an MS4A6A variant mRNA or polypeptide, the methods further comprise administering or continuing to administer the myeloid cell dysfunction agent in an amount that is the same as or less than a standard dosage amount to the subject, and/or administering an MS4A6A inhibitor or the combination of an MS4A6A inhibitor and an MS4A4A inhibitor to the subject. In embodiments where the subject has a decrease in the expression of an MS4A6A variant mRNA or polypeptide, the methods further comprise administering or continuing to administer the myeloid cell dysfunction agent in a standard dosage amount to the subject. The presence of a decrease in the expression of an MS4A6A variant mRNA or polypeptide indicates the subject has a decreased risk of developing myeloid cell dysfunction. In some embodiments, the subject has a decrease in the expression of an MS4A6A variant mRNA or polypeptide. In some embodiments, the subject does not have a decrease in the expression of an MS4A6A variant mRNA or polypeptide. In any of the embodiments described herein, the MS4A6A inhibitor is an example of a myeloid cell dysfunction agent. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

Detecting a decrease in the expression of an MS4A6A variant mRNA or polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In some embodiments, the treatment or prevention methods comprise detecting the presence or absence of an MS4A6A variant polypeptide in a biological sample from the subject. In some embodiments, when the subject does not have an MS4A6A variant polypeptide, the subject is administered a myeloid cell dysfunction agent in an amount that is the same as or less than a standard dosage amount, and/or an MS4A6A inhibitor or the combination of an MS4A6A inhibitor and an MS4A4A inhibitor. In some embodiments, when the subject has an MS4A6A variant polypeptide, the subject is administered a myeloid cell dysfunction agent in standard dosage amount.

The present disclosure also provides methods of treating a subject with a myeloid cell dysfunction agent that treats or inhibits myeloid cell dysfunction, wherein the subject has myeloid cell dysfunction or is at risk of developing myeloid cell dysfunction. The methods comprise determining whether the subject has an MS4A6A variant polypeptide by obtaining or having obtained a biological sample from the subject and performing or having performed an assay on the biological sample to determine if the subject has an MS4A6A variant polypeptide. When the subject does not have an MS4A6A variant polypeptide, the subject is administered the myeloid cell dysfunction agent in an amount that is the same as or less than a standard dosage amount, and/or an MS4A6A inhibitor or the combination of an MS4A6A inhibitor and an MS4A4A inhibitor. When the subject has an MS4A6A variant polypeptide, the subject is administered the myeloid cell dysfunction agent in a standard dosage amount. The presence of an MS4A6A variant polypeptide indicates the subject has a decreased risk of developing myeloid cell dysfunction. In some embodiments, the subject has an MS4A6A variant polypeptide. In some embodiments, the subject does not have an MS4A6A variant polypeptide.

The present disclosure also provides methods of preventing a subject from developing myeloid cell dysfunction by administering a myeloid cell dysfunction agent that prevents myeloid cell dysfunction. In some embodiments, the method comprises determining whether the subject has an MS4A6A variant polypeptide by obtaining or having obtained a biological sample from the subject and performing or having performed an assay on the biological sample to determine if the subject has an MS4A6A variant polypeptide. When the subject does not have an MS4A6A variant polypeptide, the subject is administered the myeloid cell dysfunction agent in an amount that is the same as or less than a standard dosage amount, and/or an MS4A6A inhibitor or the combination of an MS4A6A inhibitor and an MS4A4A inhibitor. When the subject has an MS4A6A variant polypeptide, the subject is administered the myeloid cell dysfunction agent in a standard dosage amount. The presence of an MS4A6A variant polypeptide indicates the subject has a decreased risk of developing myeloid cell dysfunction. In some embodiments, the subject has an MS4A6A variant polypeptide. In some embodiments, the subject does not have an MS4A6A variant polypeptide.

Detecting the presence or absence of an MS4A6A variant polypeptide in a biological sample from a subject and/or determining whether a subject has an MS4A6A variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the MS4A6A inhibitor is a small molecule. In some embodiments, the small molecule is low molecular weight (<900 daltons) organic compound.

In some embodiments, the MS4A4A inhibitor is a small molecule. In some embodiments, the small molecule is low molecular weight (<900 daltons) organic compound. In some embodiments, the MS4A4A inhibitor comprises HG-1299, a saturated fatty acid, an unsaturated fatty acid (such as, for example, decanoic acid, docosanoic acid, dodecanoic acid, eicosanoic acid, hexanoic acid, myristic acid, octadecanoic acid, octanoic acid, palmitic acid), a steroid (such as, for example, e 4-Androsten-17alpha-ol-3-one sulphate, 5-Androsten-3 Beta 17Beta-diol disulphate, 1,3,5 (10)-Estratrien-3 17Beta-diol disulphate, 1,3,5 (10)-Estratrien-3 17alpha-diol 3-sulphate, 5alpha-pregnen-3alpha-ol-20-one sulphate, 5beta-pregnen-3beta-ol-20-one sulphate, 4-pregnan-I Ibeta 21-diol-3 20-dione 21-sulphate, 4-pregnen-21-ol-3 20-ione glucosiduronate, 1,3,5 (10)-Estratrien-3 17Beta-diol 3-sulphate, 4-pregnen-1 1beta 17,21-triol3 20-dione 21-sulphate), or a compound with nitrogenous rings (such as, for example, 2,5-dimethylpyrazine, 2,6-dimethylpyrazine, 2,3-dimethylpyrazine, indole, nicotine, pyrrolidine, pyridine, quinolone), 3-aminopyrazine (3-AP), or tetramethylpyrazine.

In some embodiments, the MS4A6A inhibitor comprises an antibody, or antigen-binding fragment thereof. In some embodiments, the antibody, or antigen-binding fragment thereof, binds specifically to human MS4A6A. In some embodiments, the antibody is a fully human monoclonal antibody (mAb), or antigen-binding fragment thereof, that specifically binds and neutralizes, inhibits, blocks, abrogates, reduces, or interferes with, at least one activity of MS4A6A, in particular, human MS4A6A. In some embodiments, an antibody or fragment thereof can neutralize, inhibit, block, abrogate, reduce, or interfere with, an activity of MS4A6A by binding to an epitope of MS4A6A that is directly involved in the targeted activity of MS4A6A. In some embodiments, an antibody or fragment thereof can neutralize, inhibit, block, abrogate, reduce, or interfere with, an activity of MS4A6A by binding to an epitope of MS4A6A that is not directly involved in the targeted activity of MS4A6A, but the antibody or fragment binding thereto sterically or conformationally inhibits, blocks, abrogates, reduces, or interferes with, the targeted activity of MS4A6A. In some embodiments, an antibody or fragment thereof binds to an epitope of MS4A6A that is not directly involved in the targeted activity of MS4A6A (i.e., a non-blocking antibody), but the antibody or fragment binding thereto results in the enhancement of the clearance of MS4A6A from the circulation, compared to the clearance of MS4A6A in the absence of the antibody or fragment thereof, thereby indirectly inhibiting, blocking, abrogating, reducing, or interfering with, an activity of MS4A6A. Clearance of MS4A6A from the circulation can be particularly enhanced by combining two or more different non-blocking antibodies that do not compete with one another for specific binding to MS4A6A. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the MS4A4A inhibitor comprises an antibody, or antigen-binding fragment thereof. In some embodiments, the antibody, or antigen-binding fragment thereof, binds specifically to human MS4A4A. In some embodiments, the antibody is a fully human monoclonal antibody (mAb), or antigen-binding fragment thereof, that specifically binds and neutralizes, inhibits, blocks, abrogates, reduces, or interferes with, at least one activity of MS4A4A, in particular, human MS4A4A. In some embodiments, an antibody or fragment thereof can neutralize, inhibit, block, abrogate, reduce, or interfere with, an activity of MS4A4A by binding to an epitope of MS4A4A that is directly involved in the targeted activity of MS4A4A. In some embodiments, an antibody or fragment thereof can neutralize, inhibit, block, abrogate, reduce, or interfere with, an activity of MS4A4A by binding to an epitope of MS4A4A that is not directly involved in the targeted activity of MS4A4A, but the antibody or fragment binding thereto sterically or conformationally inhibits, blocks, abrogates, reduces, or interferes with, the targeted activity of MS4A4A. In some embodiments, an antibody or fragment thereof binds to an epitope of MS4A4A that is not directly involved in the targeted activity of MS4A4A (i.e., a non-blocking antibody), but the antibody or fragment binding thereto results in the enhancement of the clearance of MS4A4A from the circulation, compared to the clearance of MS4A4A in the absence of the antibody or fragment thereof, thereby indirectly inhibiting, blocking, abrogating, reducing, or interfering with, an activity of MS4A4A. Clearance of MS4A4A from the circulation can be particularly enhanced by combining two or more different non-blocking antibodies that do not compete with one another for specific binding to MS4A4A. The antibodies can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment thereof specifically binds to MS4A6A or MS4A4A with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-MS4A6A or anti-MS4A4A antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made to modify complement dependent cytotoxicity (CDC).

In some embodiments, the anti-MS4A6A antibodies include, but are not limited to, the antibodies disclosed in PCT Publications WO 2019/152715 and WO 2019/152706. In some embodiments, the MS4A6A inhibitor can be any MS4A6A inhibitor disclosed in PCT Publication WO 2017/143036.

In some embodiments, the anti-MS4A4A antibodies include, but are not limited to, anti-human MS4A4A antibody (Abcam, Catalog No. ab67134) and anti-human MS4A4A antibody (Sigma-Aldrich, Catalog No. HPA029323) (Li et al., Gut, 2023, 1-14), or any of those disclosed in, for example, WO 2019/152715, WO 2019/152706, and WO 2021/022083.

The present disclosure also provides compositions comprising a combination of an antibody or antigen-binding fragment thereof and a myeloid cell dysfunction agent.

In some embodiments, the myeloid cell dysfunction agents include, but are not limited to, acetylcholinesterase inhibitors (such as, for example, tacrine, rivastigmine, galantamine, and donepezil), memantine, EGb 761, aducanumab, cerebrolysin, idebenone, lecanemab, olanzapine, prazosin, thioridazine, and trazodone. In some embodiments, the myeloid cell dysfunction agent comprises an acetylcholinesterase inhibitor. In some embodiments, the acetylcholinesterase inhibitor comprises tacrine. In some embodiments, the acetylcholinesterase inhibitor comprises rivastigmine. In some embodiments, the acetylcholinesterase inhibitor comprises galantamine. In some embodiments, the acetylcholinesterase inhibitor comprises donepezil. In some embodiments, the myeloid cell dysfunction agent comprises memantine. In some embodiments, the myeloid cell dysfunction agent comprises EGb 761. In some embodiments, the myeloid cell dysfunction agent comprises aducanumab. In some embodiments, the myeloid cell dysfunction agent comprises cerebrolysin. In some embodiments, the myeloid cell dysfunction agent comprises idebenone. In some embodiments, the myeloid cell dysfunction agent comprises lecanemab. In some embodiments, the myeloid cell dysfunction agent comprises olanzapine. In some embodiments, the myeloid cell dysfunction agent comprises prazosin. In some embodiments, the myeloid cell dysfunction agent comprises thioridazine. In some embodiments, the myeloid cell dysfunction agent comprises trazodone.

In some embodiments, the myeloid cell dysfunction agent can be combined with an MS4A6A inhibitor or a combination of an MS4A6A inhibitor and an MS4A4A inhibitor.

In some embodiments, the dose of the myeloid cell dysfunction agents that treat, prevent, or inhibit myeloid cell dysfunction can be decreased by about 10%, by about 20%, by about 30%, by about 40%, by about 50%, by about 60%, by about 70%, by about 80%, or by about 90% for subjects that are heterozygous for an MS4A6A variant nucleic acid molecule or MS4A6A reference (i.e., a less than the standard dosage amount) compared to subjects that are homozygous for an MS4A6A variant nucleic acid molecule (who may receive a standard dosage amount). In some embodiments, the dose of the myeloid cell dysfunction agents that treat, prevent, or inhibit myeloid cell dysfunction can be decreased by about 10%, by about 20%, by about 30%, by about 40%, or by about 50%. In some embodiments, the dose of the myeloid cell dysfunction agents that treat, prevent, or inhibit myeloid cell dysfunction can be decreased by about 10%, by about 20%, by about 30%, by about 40%, by about 50%, by about 60%, by about 70%, by about 80%, or by about 90% for subjects that are heterozygous for an MS4A6A variant nucleic acid molecule or MS4A6A reference compared to subjects that are MS4A6A reference. In addition, subjects that are heterozygous for an MS4A6A variant nucleic acid molecule or MS4A6A reference can be administered the myeloid cell dysfunction agents less frequently compared to subjects that are heterozygous for the MS4A6A variant nucleic acid molecule.

Administration of the myeloid cell dysfunction agents that treat, prevent, or inhibit myeloid cell dysfunction and/or MS4A6A inhibitors and/or MS4A4A inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the myeloid cell dysfunction agents and/or MS4A6A inhibitors and/or MS4A4A inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in myeloid cell dysfunction, a decrease/reduction in the severity of myeloid cell dysfunction (such as, for example, a reduction or inhibition of development of myeloid cell dysfunction), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to myeloid cell dysfunction, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of myeloid cell dysfunction development/progression (such as, for example, a complete or partial avoidance/inhibition or a delay), and an increased survival time of the affected host animal, following administration of a therapeutic protocol. Treatment of myeloid cell dysfunction encompasses the treatment of a subject already diagnosed as having any form of myeloid cell dysfunction at any clinical stage or manifestation, the delay of the onset or evolution or aggravation or deterioration of the symptoms or signs of myeloid cell dysfunction, and/or preventing and/or reducing the severity of myeloid cell dysfunction.

In some embodiments, the MS4A6A inhibitor and/or MS4A4A inhibitor, and the myeloid cell dysfunction agent are disposed within a pharmaceutical composition. In some embodiments, the MS4A6A inhibitor and/or MS4A4A inhibitor is disposed within a first pharmaceutical composition and the myeloid cell dysfunction agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure also provides methods of identifying a subject having an increased risk of developing myeloid cell dysfunction. In some embodiments, the method comprises determining or having determined in a biological sample obtained from the subject the presence or absence of an MS4A6A variant nucleic acid molecule (such as a genomic nucleic acid molecule, mRNA molecule, and/or cDNA molecule). When the subject lacks an MS4A6A variant nucleic acid molecule (i.e., the subject is genotypically categorized as MS4A6A reference), then the subject has an increased risk of developing myeloid cell dysfunction. When the subject has an MS4A6A variant nucleic acid molecule (i.e., the subject is heterozygous or homozygous for an MS4A6A variant nucleic acid molecule), then the subject has a decreased risk of developing myeloid cell dysfunction. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

Having a single copy of an MS4A6A variant nucleic acid molecule is more protective of a subject from developing myeloid cell dysfunction than having no copies of an MS4A6A variant nucleic acid molecule. Without intending to be limited to any particular theory or mechanism of action, it is believed that a single copy of an MS4A6A variant nucleic acid molecule (i.e., heterozygous for an MS4A6A variant nucleic acid molecule) is protective of a subject from developing myeloid cell dysfunction and it is also believed that having two copies of an MS4A6A variant nucleic acid molecule (i.e., homozygous for an MS4A6A variant nucleic acid molecule) may be more protective of a subject from developing myeloid cell dysfunction, relative to a subject with a single copy. Thus, in some embodiments, a single copy of an MS4A6A variant nucleic acid molecule may not be completely protective, but instead, may be partially or incompletely protective of a subject from developing myeloid cell dysfunction. While not desiring to be bound by any particular theory, there may be additional factors or molecules involved in the development of myeloid cell dysfunction that are still present in a subject having a single copy of an MS4A6A variant nucleic acid molecule, thus resulting in less than complete protection from the development of myeloid cell dysfunction.

Determining whether a subject has an MS4A6A variant nucleic acid molecule in a biological sample from a subject and/or determining whether a subject has an MS4A6A variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing myeloid cell dysfunction, the subject is administered a myeloid cell dysfunction agent, and/or an MS4A6A inhibitor or the combination of an MS4A6A inhibitor and an MS4A4A inhibitor, as described herein. For example, when the subject is MS4A6A reference, and therefore has an increased risk of developing myeloid cell dysfunction, the subject is administered a myeloid cell dysfunction agent in an amount that is the same as or less than a standard dosage amount, and/or is administered an MS4A6A inhibitor or the combination of an MS4A6A inhibitor and an MS4A4A inhibitor. In some embodiments, when the subject is heterozygous for an MS4A6A variant nucleic acid molecule, the subject is administered the myeloid cell dysfunction agent in an amount that is the same as or less than a standard dosage amount, and/or is administered an MS4A6A inhibitor or the combination of an MS4A6A inhibitor and an MS4A4A inhibitor. In some embodiments, when the subject is homozygous for an MS4A6A variant nucleic acid molecule, the subject is administered a myeloid cell dysfunction agent in a standard dosage amount. In some embodiments, the subject is MS4A6A reference. In some embodiments, the subject is heterozygous for an MS4A6A variant nucleic acid molecule. In some embodiments, the subject is homozygous for an MS4A6A variant nucleic acid molecule.

The present disclosure also provides methods of determining a subject's aggregate burden, or risk score, of having two or more MS4A6A variant nucleic acid molecules, and/or two or more MS4A6A variant polypeptides associated with a decreased risk of developing myeloid cell dysfunction. The aggregate burden is the sum of two or more genetic variants that can be carried out in an association analysis with myeloid cell dysfunction. In some embodiments, the subject is homozygous for one or more MS4A6A variant nucleic acid molecules associated with a decreased risk of developing myeloid cell dysfunction. In some embodiments, the subject is heterozygous for one or more MS4A6A variant nucleic acid molecules associated with a decreased risk of developing myeloid cell dysfunction. When the subject has a lower aggregate burden, the subject has an increased risk of developing myeloid cell dysfunction, and the subject is administered or continued to be administered the myeloid cell dysfunction agent in an amount that is the same as or less than the standard dosage amount, and/or an MS4A6A inhibitor. When the subject has a higher aggregate burden, the subject has a decreased risk of developing myeloid cell dysfunction and the subject is administered or continued to be administered the myeloid cell dysfunction agent in a standard dosage amount. The higher the aggregate burden, the lower the risk of developing myeloid cell dysfunction.

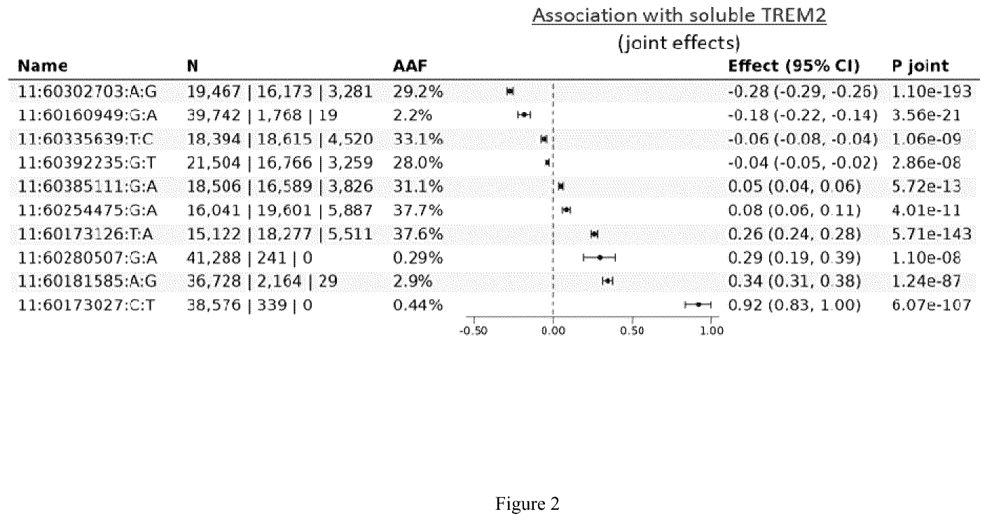

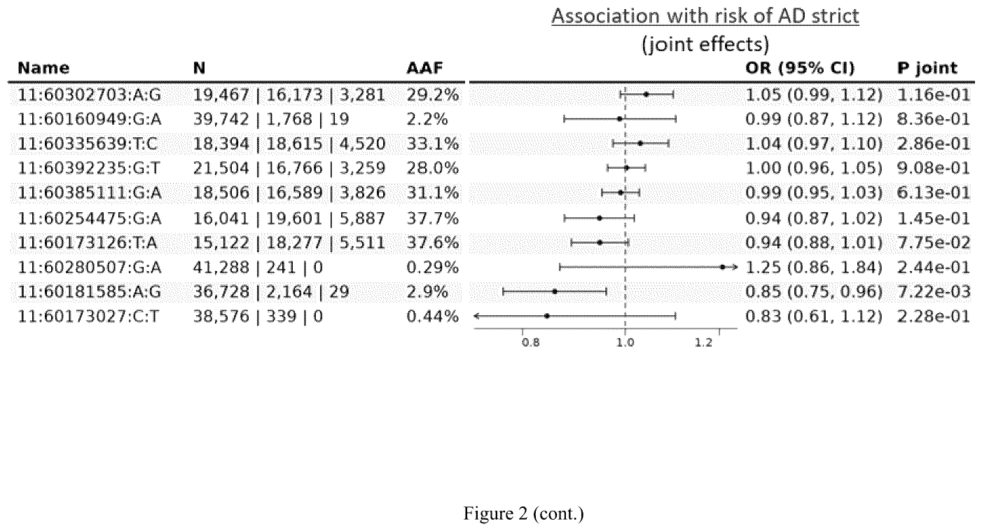

In some embodiments, a subject's aggregate burden of having any two or more MS4A6A variant nucleic acid molecules represents a weighted sum of a plurality of any of the MS4A6A variant nucleic acid molecules and their non-coding regulators, including but not limited to, the genetic variations shown in FIG. 2 disclosed herein. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the MS4A6A gene, where the genetic burden is the number of alleles multiplied by the association estimate with myeloid cell dysfunction or related outcome (e.g., sTREM levels or Alzheimer's disease) for each allele (i.e., a weighted polygenic burden score). In some embodiments, when the subject has an aggregate burden higher than a desired threshold score, the subject has a decreased risk of developing myeloid cell dysfunction. In some embodiments, when the subject has an aggregate burden lower than a desired threshold score, the subject has an increased risk of developing myeloid cell dysfunction.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with myeloid cell dysfunction in the top 10%, top 20%, top 30%, top 40%, or top 50% of p-value range for the association. In some embodiments, each of the identified genetic variants comprise the genetic variants having association with myeloid cell dysfunction with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with myeloid cell dysfunction with p-value of less than 5×10⁻⁸. In some embodiments, the identified genetic variants comprise genetic variants having association with myeloid cell dysfunction in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

In embodiments where the aggregate burden is determined for MS4A6A genetic variants associated with myeloid cell dysfunction, then the aggregate burden represents a subject's risk score for developing myeloid cell dysfunction. In some embodiments, the aggregate burden or risk score includes the MS4A6A variant genomic nucleic acid molecule that comprises any one or more of the genetic variations in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs), as well as any other coding or non coding (i.e., intronic or 5′ UTR) genetic variations, or an mRNA molecule produced therefrom, or a cDNA molecule produced from the mRNA molecule. In some embodiments, a subject's aggregate burden can be determined for MS4A6A genetic variants associated with myeloid cell dysfunction in combination with additional genetic variants for other genes also associated with myeloid cell dysfunction to produce a polygenic risk score (PRS) for developing myeloid cell dysfunction. In some embodiments, the PRS includes the MS4A6A variant genomic nucleic acid molecule that comprises any one or more of the genetic variations in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides methods of detecting the presence or absence of an MS4A6A variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any MS4A6A variant nucleic acid molecule, preliminary processing designed to isolate or enrich the biological sample for the genomic DNA can be employed. A variety of techniques may be used for this purpose. When detecting the level of any MS4A6A variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an MS4A6A variant nucleic acid molecule in a subject comprises performing a sequence analysis on a biological sample obtained from the subject to determine whether an MS4A6A genomic nucleic acid molecule in the biological sample, and/or an MS4A6A mRNA molecule in the biological sample, and/or an MS4A6A cDNA molecule produced from an mRNA molecule in the biological sample, is present in the sample. In some embodiments, the methods detect the MS4A6A variant genomic nucleic acid molecule that comprises any one or more of the genetic variations in Table 1, such as: 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs), as well as any other coding or non coding (i.e., intronic or 5′ UTR) genetic variations, or an mRNA molecule produced therefrom, or a cDNA molecule produced from the mRNA molecule.

In some embodiments, the methods of detecting the presence or absence of an MS4A6A variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an MS4A6A genomic nucleic acid molecule or mRNA molecule, and if mRNA, optionally reverse transcribing the mRNA into cDNA. Such assays can comprise, for example determining the identity of these positions of the particular MS4A6A nucleic acid molecule. In some embodiments, the method is an in vitro method.

In some embodiments, the determining step, detecting step, or sequence analysis comprises sequencing at least a portion of the nucleotide sequence of the MS4A6A genomic nucleic acid molecule, the MS4A6A mRNA molecule, or the MS4A6A cDNA molecule in the biological sample that comprises a genetic variation compared to the corresponding MS4A6A reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an MS4A6A genomic nucleic acid molecule is analyzed. In some embodiments, only an MS4A6A mRNA is analyzed. In some embodiments, only an MS4A6A cDNA obtained from the MS4A6A mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an MS4A6A variant genomic sequence, variant mRNA sequence, or variant cDNA sequence and not the corresponding MS4A6A reference sequence under stringent conditions and determining whether hybridization has occurred.

In some embodiments, the determining step, detecting step, or sequence analysis comprises: a) amplifying at least a portion of the MS4A6A nucleic acid molecule that encodes the MS4A6A polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an MS4A6A variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to MS4A6A variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to MS4A6A variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the MS4A6A variant nucleic acid molecules disclosed herein. The primers described herein can be used to amplify any MS4A6A variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an MS4A6A reference genomic nucleic acid molecule, an MS4A6A reference mRNA molecule, and/or an MS4A6A reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6Xhis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides myeloid cell dysfunction agents that treat, prevent, or inhibit myeloid cell dysfunction for use in the treatment or prevention of myeloid cell dysfunction in a subject having an MS4A6A variant nucleic acid molecule. Any of the myeloid cell dysfunction agents that treat, prevent, or inhibit myeloid cell dysfunction described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides uses of myeloid cell dysfunction agents that treat, prevent, or inhibit myeloid cell dysfunction for use in the preparation of a medicament for treating or preventing myeloid cell dysfunction in a subject having an MS4A6A variant nucleic acid molecule. Any of the myeloid cell dysfunction agents that treat, prevent, or inhibit myeloid cell dysfunction described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides MS4A6A inhibitors for use in the treatment or prevention of myeloid cell dysfunction in a subject that is MS4A6A reference or is heterozygous for an MS4A6A variant nucleic acid molecule. Any of the MS4A6A inhibitors described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides MS4A6A inhibitors in the preparation of a medicament for treating or preventing myeloid cell dysfunction in a subject that is MS4A6A reference or is heterozygous for an MS4A6A variant nucleic acid molecule. Any of the MS4A6A inhibitors described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides the combination of MS4A6A inhibitors and MS4A4A inhibitors for use in the treatment or prevention of myeloid cell dysfunction in a subject that is MS4A6A reference or is heterozygous for an MS4A6A variant nucleic acid molecule. Any of the MS4A6A inhibitors and MS4A4A inhibitors described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

The present disclosure also provides the combination of MS4A6A inhibitors and MS4A4A inhibitors in the preparation of a medicament for treating or preventing myeloid cell dysfunction in a subject that is MS4A6A reference or is heterozygous for an MS4A6A variant nucleic acid molecule. Any of the MS4A6A inhibitors and MS4A4A inhibitors described herein can be used herein. Any of the MS4A6A variant nucleic acid molecules disclosed herein can be used herein. In some embodiments, the MS4A6A variant nucleic acid molecule is a MS4A6A variant genomic nucleic acid molecule that comprises any one or more of the genetic variations in Table 1, such as: 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs), as well as any other coding or non coding (i.e., intronic or 5′ UTR) genetic variations, or an mRNA molecule produced therefrom, or a cDNA molecule produced from the mRNA molecule.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: General Methods

### Sample Genotyping

The UK Biobank was genotyped using two different genotyping arrays, the Applied Biosystems UK BiLEVE Axiom Array by Affymetrix and the closely related Applied Biosystems UK Biobank Axiom Array. For whole exome sequencing, DNA libraries were created by enzymatically shearing DNA to a mean fragment size of 200 base pairs, and a common Y-shaped adapter was ligated to all DNA libraries. Unique, asymmetric 10-base-pair barcodes were added to the DNA fragment during library amplification to facilitate multiplexed exome capture and sequencing. Equal amounts of sample were pooled before overnight exome capture, with a slightly modified version of IDT's xGen probe library. The following cohorts: UPENN-PMBB, SINAI and INDIANA-CHALASANI were genotyped using the illumina Global Screening Array (GSA) genotyping array. The GHS samples were genotyped using either the Illumina Infinium OmniExpressExome or the GSA. Whole exome sequencing was performed for the samples from the following cohots UPENN-PMBB, SINAI, INDIANA-CHALASANI and GHS following similar procedures to those described fro the UK-Biobank. The samples from the remaining cohorts, UCLA, MAYO-CLINIC and COLORADO were genotyped using whole exome and targeted genomic sequencing using the twist diversity SNP panel, followed by the multipoint refinement using GLIMPSE. Within each cohort, standard quality-control procedures (see below) were followed to retain only high-quality genotyped variants, which were then used for imputing common variants using the TOPMed LD reference panel.

### Genotype Quality Control

Stringent and standardized pre-imputation quality control was performed for each cohort. Briefly, the following filters and checks were implemented including, removal of variants with missingness >0.01; removal of samples with missingness >0.1; removal variants with significant deviations from Hardy-Weinberg equilibrium (p<1e-15). For this study, post imputation QC included retaining common variants (minimum MAF>1%) and MaCH 3 R2>0.1. For cohorts for which genotype by sequencing was used, a specialized multipoint refinement (GLIMPSE) based variant caller was used to increase the call rate and concordance despite differential coverage across sites.

### Alzheimer's Disease Phenotype Definitions

Three definitions were used for phenotypes related to Alzheimer's disease. Alzheimer's disease strict definition: Cases were those who had ICD-10 G30 diagnosis, or dementia in AD (F00); Broad Alzheimer's Disease definition: cases were those who had ICD-10 G30 diagnosis, or dementia in AD (F00), or other primary non-demyelinating, non-vascular degenerative conditions (G310|G311|F00|F02|F03); Broad Alzheimer's disease plus family history: cases were those who had ICD-10 G30 diagnosis, or dementia in AD (F00), or other primary non-demyelinating, non-vascular degenerative conditions (G310|G311| F00| F02| F03) or a first degree relative with a history of Alzheimer's disease. Controls were those who had no neurodegenerative disorders (G20-G26, G30-G32, G35-G37) nor family history.

Measurement of sTREM and Other Proteins

The UK Biobank plasma proteomics project is a multi-institution collaboration that quantified the levels of 2,923 proteins across 54,219 participants of the UK-Biobank. Relative protein abundance was measured using the Olink Explore 3072 platform. Association analyses for plasma sTREM2, including GWAS (see below) as well as logistic regressions assessing sTREM2 and Alzheimer's disease, were performed within individuals of European ancestry and including age, sex, genetic principal components and the following 9 relevant covariates: Current smoking, Whole body fat mass, Cystatin C, Alanine aminotransferase, Apolipoprotein A, LDL direct, Albumin, Number of medications, Aspartate aminotransferase.

### Genome Wide Association Studies

For binary traits, association analyses in each cohort were performed using the genome-wide Firth logistic regression test implemented in REGENIE v3.2.8. For quantitative traits such as plasma sTREM2, a linear mixed effects model regression implemented in in REGENIE v3.2.8 was used for association testing. In step 1 of REGENIE, the variants derived from TOPMed imputation with MAF>1%, 10% missingness, Hardy-Weinberg equilibrium test p>10⁻¹⁵ and linkage disequilibrium (LD) pruning (1,000 variant windows, 100 variant sliding windows and r²<0.9) were included. The association model used in REGENIE included as covariates (1) age, age squared, sex, age-x-sex, and age squared-x-sex; (2) 10 ancestry-informative principal components (PCs) derived from the analysis of a stricter set of LD-pruned common variants from the imputed data; (3) for the analysis of GHS, we additionally included genotyping batch (4 batches) as covariates; (4) for the analysis of SINAI, we additionally included two covariates F_MISS and miss_bin, where F_MISS was the direct measure of missingness rates observed in imputation and miss_bin was binarization of the F_MISS variable at missingness=0.0017; (5) for the analysis of UPENN-PMBB, we additionally included one batch indicator. Where relevant, associations for specific groups of variables were repeated including all of the variants within the group in a sinlge model to estimate the variants joint effect and statistical association with a given phenotype.

Identification of Genetic Variants in the MS4 Locus Controlling sTREM2 Levels

To detect conditionally independent genetic variants associated with plasma STREM2 near the MS4 locus we ran an iterative conditional analysis. This approach is akin to a stepwise regression whereby top independent signals in a GWAS are identified by distance clumping. Once these variants are identified a subsequent round of GWAS is performed, now adjusting for the samples' genotypes at these top signals. This process was repeated until there we no genome-wide significant variants left.

### STREM2 Genetic Risk Score Estimation

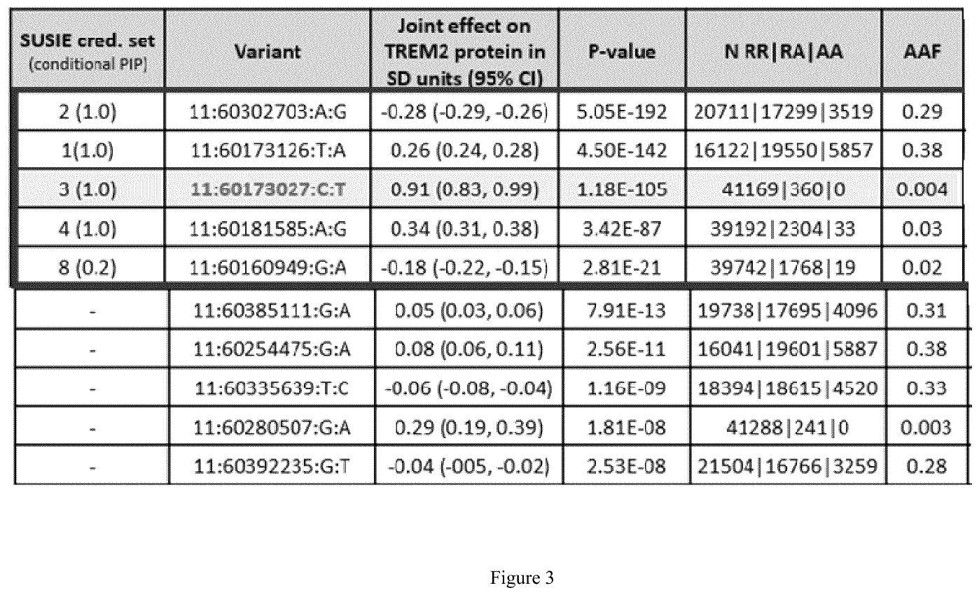

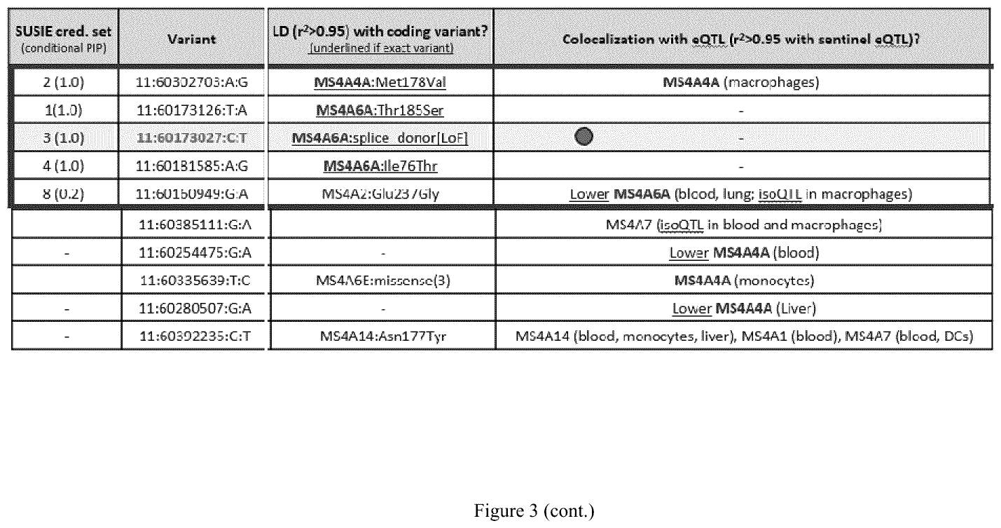

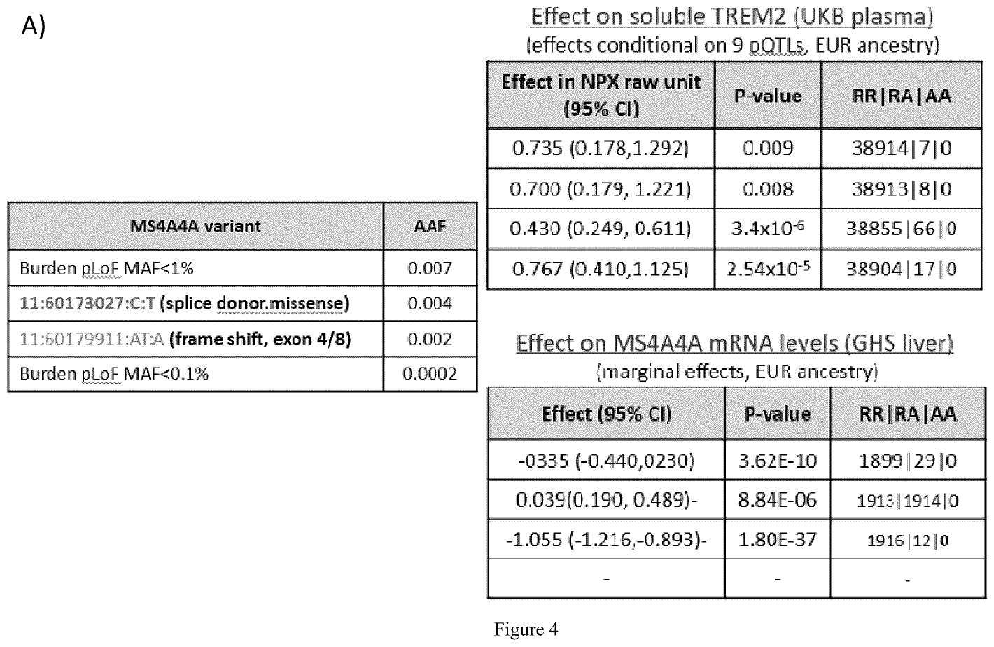

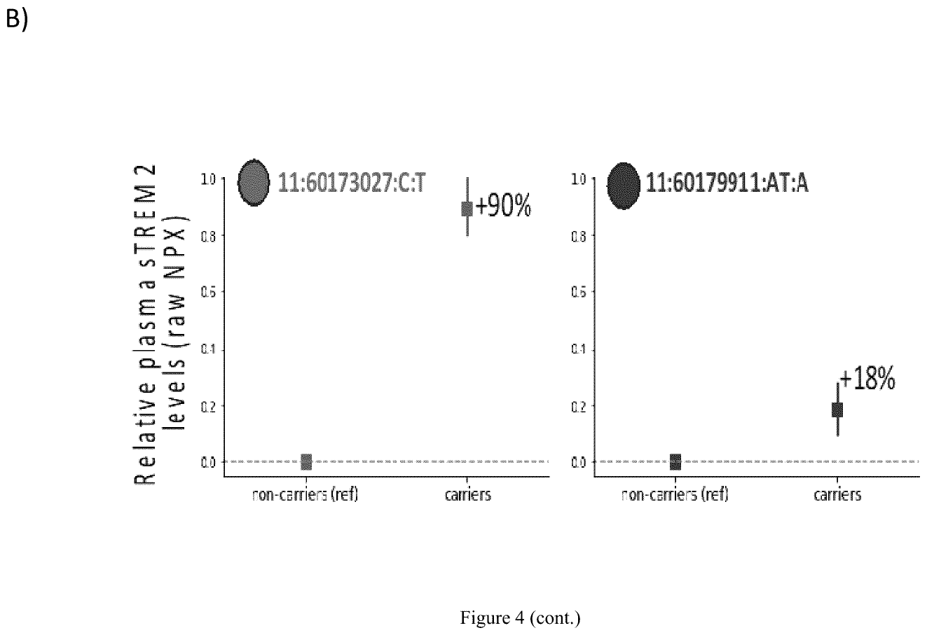

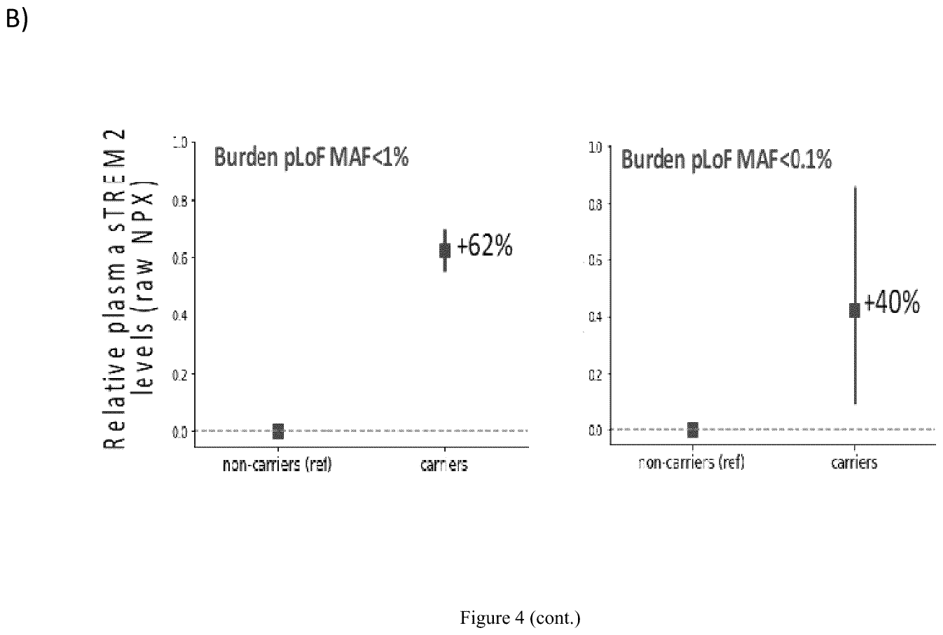

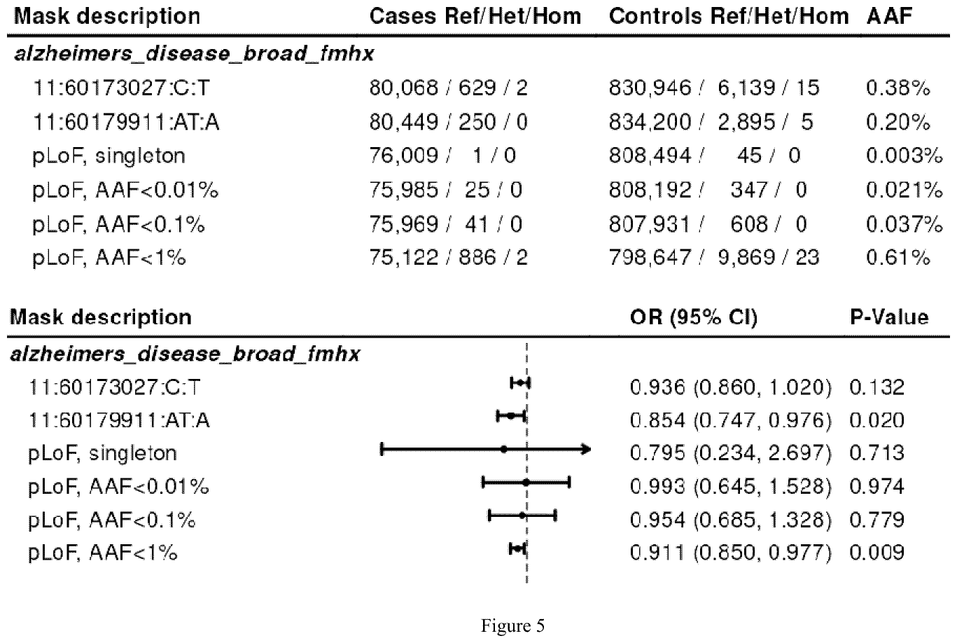

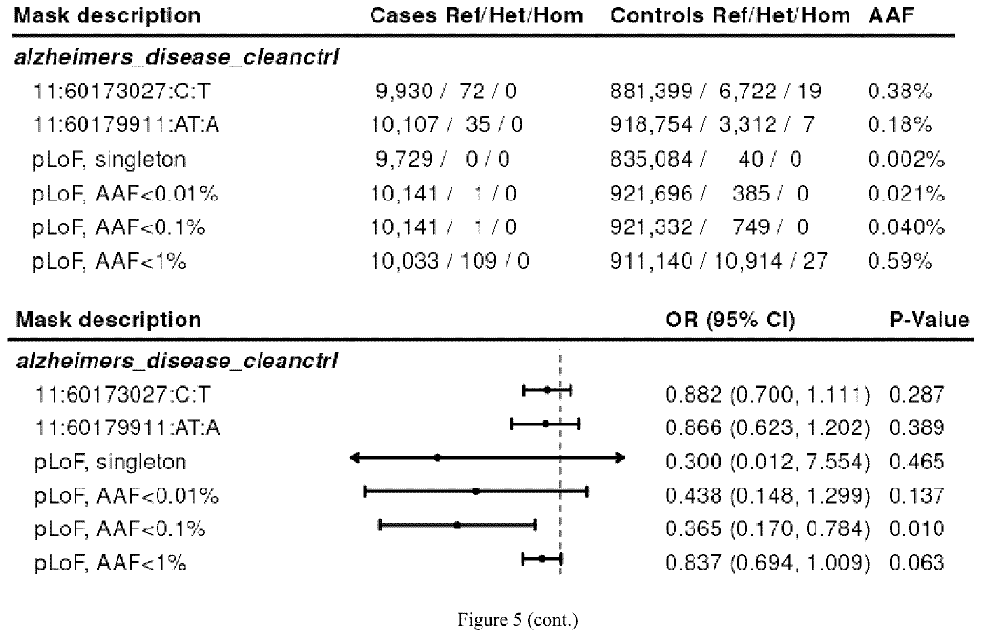

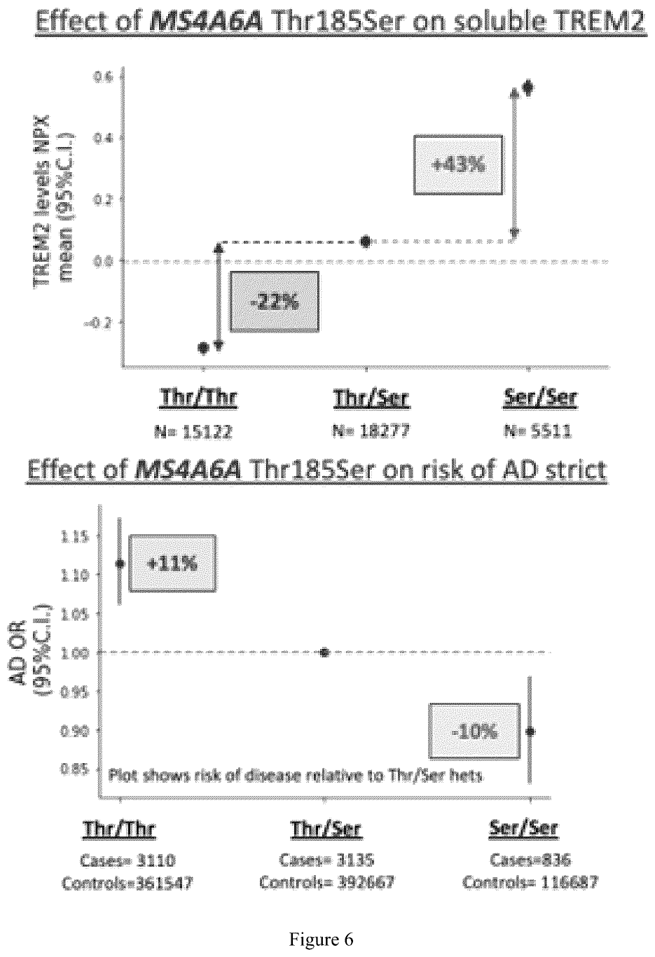

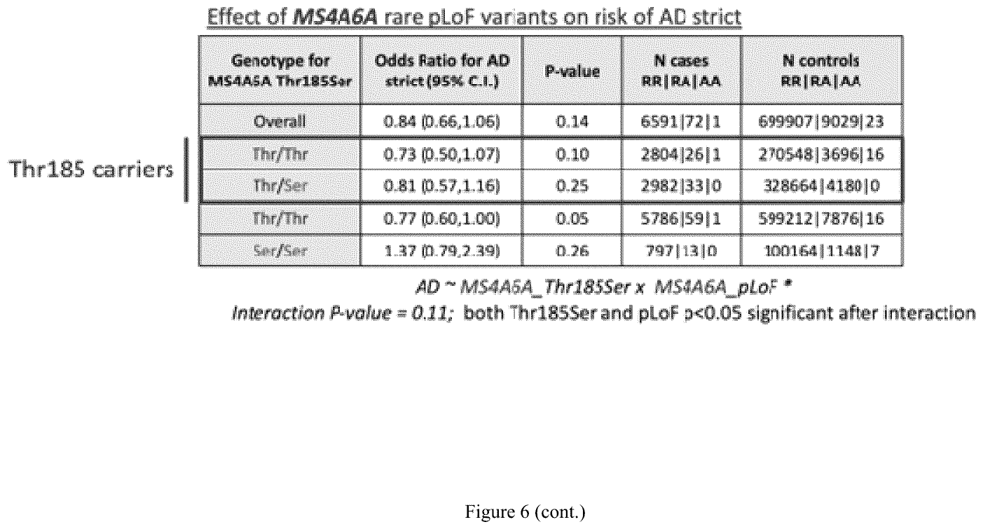

The results from the iterative conditional analyses were used to define independent significant variants in the MS4 locus associated with abundance of plasma STREM2 (FIG. 3). We estimated the joint effects of these variants within a single regression model accounting for the same covariates and random effects as the discovery GWAS. These joint effect sizes were then used to estimate genetic risk scores (GRS) across all the cohorts for which genetic and phenotypic data was available. Briefly, a GRS is estimated for each participant by performing a sum of their genotypes weighted by their allelic effect sizes. Analyses correlating the sTREM2 GRS with quantitative or binary traits were perfomed using linear and logistic regressions respectively. Morevoer, the same covariates as for the GWAS were used in all regression analyses with GRS for sTREM2 unless otherwise stated.

### Example 2: Genome-Wide Association Studies for Alzheimer's Disease Phenotypes

Genome-wide association studies for Alzheimer's disease phenotypes and plasma soluble TREM2 were performed as described above. The plasma sTREM2 GWAS results was used to construct a plasma sTREM2 GRS, based on ten conditionally independent genetic variants in the MS4 locus. The sample was split into plasma sTREM2 quintiles (bottom 20%, 20 to 40%, 40 to 60%, 60 to 80% and top 80%) and quantile regressions and logistic regressions were emlyed to explore the relationship between GRS for sTREM2 and Alzheimer's disease. Altogether, the results suggest that a biological mechanism increasing plasma sTREM2 is associated with lower risk for Alzheimer's disease. This was observed both by studying measured sTREM2 and risk for Alzheimer's disease as well as at the genetic level using a plasma sTREM2 genetic risk score. The exome association results demonstrate a relationship between lower MS4A6A activity or function and higher sTREM. Consistently, evidence was found for loss of function variants (or burden of variants) of MS4A6A to be associated with lower risk for developing Alzheimer's disease. Importantly, the results suggested that the protective direction of effect of MS4A6A loss of function was only apparent in samples that were carriers of the reference, or pathogenic (i.e., those reducing plasma sTREM) alleles. This leads us to hypothesize that patient stratification and response to MS4A6A silencing or blocking is possible based on MS4A6A genotypes. Experimental results in vitro showed increased TREM2 signaling upon silencing of MS4A6A, further supporting the human genetic observations.

### Example 3: Inhibition of MS4A6A

### THP1 Cell Lines

WT and TREM2 KO THP1 cells from Abcam (ab271147 and ab269489, respectively) were cultured in complete media (RPMI+10% FBS+0.05 mM β-mercaptoethanol+1% Penicillin/Streptomycin/L-glutamine).

siRNA Knockdown of MS4A6A

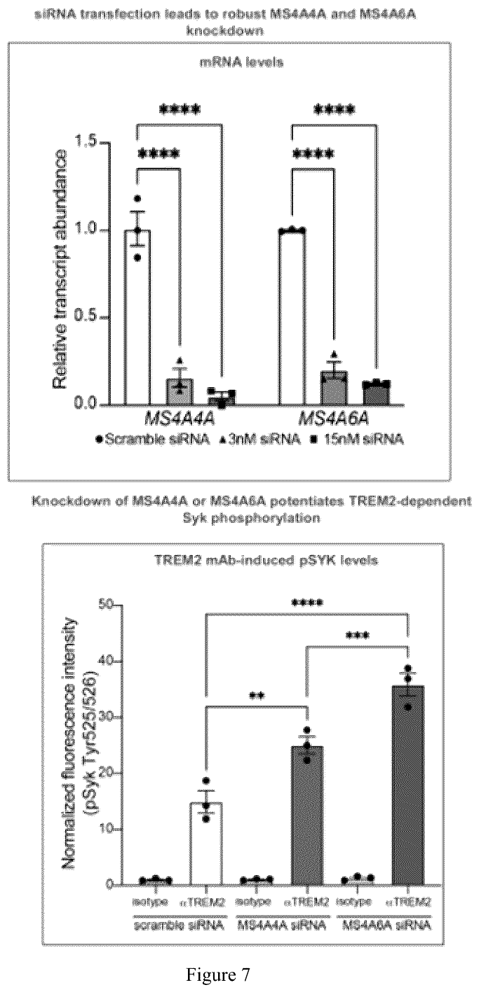

THP1 cells were differentiated for 72 hours in complete media containing 25 nM phorbol 12-myristate 13-acetate (PMA). After differentiation, media cells were allowed 24-hour recovery in complete media without PMA before transfection. Transfection was performed according to the manufacturer's guidelines using control or silencer select siRNAs targeting genes MS4A4A (s27981, ThermoFisher Scientific) and MS4A6A (s224607, ThermoFisher Scientific). In brief, diluted siRNAs were mixed with lipofectamine RNAiMax transfection reagent (ThermoFisher Scientific, 13778150) in OptiMEM reduced serum media, allowed to form lipid-siRNA complex, and then added to THP1 cells. Transfection media was removed and replaced with complete media after 24 hours, and cells were processed for RNA isolation or for pSyk assay after 48 hours. Quantitative real time PCR analyses showed dose-dependent knockdown of MS4A4A and MS4A6A in THP1 cells treated with the respective targeting siRNAs compared to scramble siRNA treated cells. These results show that the siRNA treatment paradigm achieves >80% MS4A4A and MS4A6A knockdown, thus providing a platform for interrogating effects of gene knockdown on TREM2 function (FIG. 7).

pSYK AlphaLISA

At 72 hours post siRNA transfection, anti-TREM2 (R&D Systems, MAβ 17291) or isotype control (R&D Systems, MAB0061) antibodies were added to differentiated THP1 cells for 20 minutes at 4° C. Cells were then twice washed with cold PBS, followed by 10-minute incubation with crosslinking antibody (Jackson Labs, 112-001-008) at 37° C. Cells were again washed twice with cold PBS then AlphaLISA assay was performed according to manufacturer instruction (PerkinElmer, ALSU-PSYK-B-HV). In brief, cells were lysed and successively incubated in donor—and acceptor-bead complexes for 1 and 2 hours, respectively. Fluorescence intensity was measured then on PerkenElmer Envision plate reader (FIG. 7). MS4A6A siRNA treated cells show augmented TREM2 agonist antibody induced SYK phosphorylation compared to scramble siRNA treated cells leads. Similar results were obtained in MS4A4A siRNA treated cells, albeit to a lesser extent than MS4A6A treated cells. Taken together, these results indicate that MS4A4A/6A antagonize TREM2 function, and support the notion that knockdown of MS4A4A/6A promotes increased TREM2-dependent functions.

### Ms4a6* Triple KO Mice

Human MS4A6A has 3 orthologous genes in mice: Ms4a6b (53% amino acid identity), Ms4a6c (45% identity), and Ms4a6d (52% identity). All three genes were knocked out, as well as two additional genes (Ms4a4d, Gm8369) using CRISPR-based collapse of the region spanning Ms4a6b/c/d in mice. These mice are referred to as Ms4a6* triple KO mice. Ms4a6* triple KO mice are viable and have no obvious abnormalities.

### Induction of Rapid a Model

In order to induce Alzheimer's disease (AD)-like pathology in mice, co-delivered 2 AAV9s, one expressing human APP695 with three familial AD mutations (KM670/671NL+E693G+I716F) and another expressing human PSEN1 with two familial AD mutations (M146L+L286V) were used. Both viruses were driven by the human Synapsin promoter. Two months post-intrahippocampal injections of these 2 viruses, mice developed Aβ deposits, microglial and astrocyte activation, and neurite damage. This model builds on previously described AAV models delivering only hAPP with familial AD mutations (PMID 19794916), where Aβ plaques were shown six months post-injection.

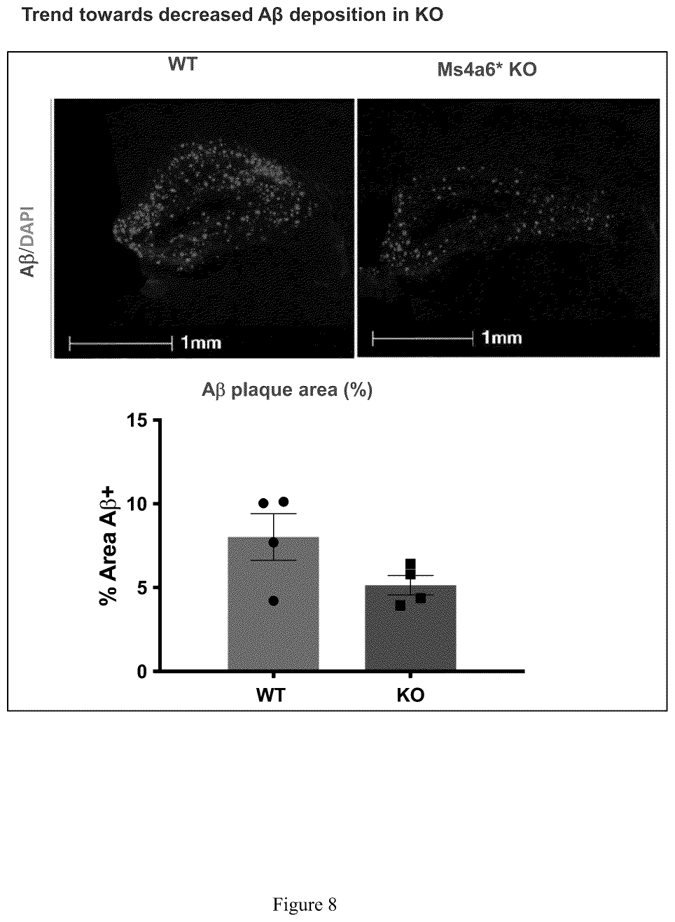

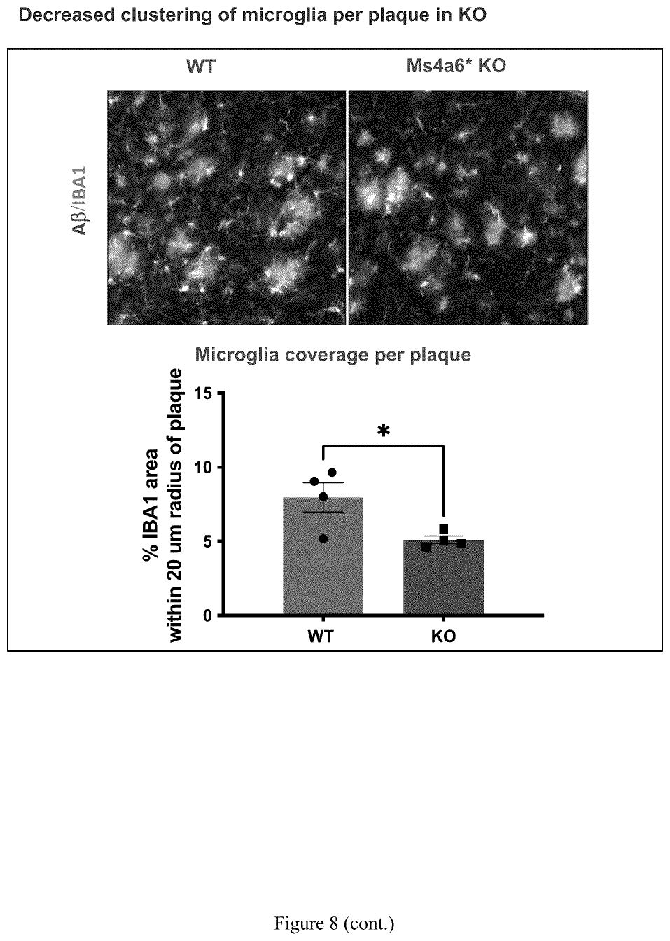

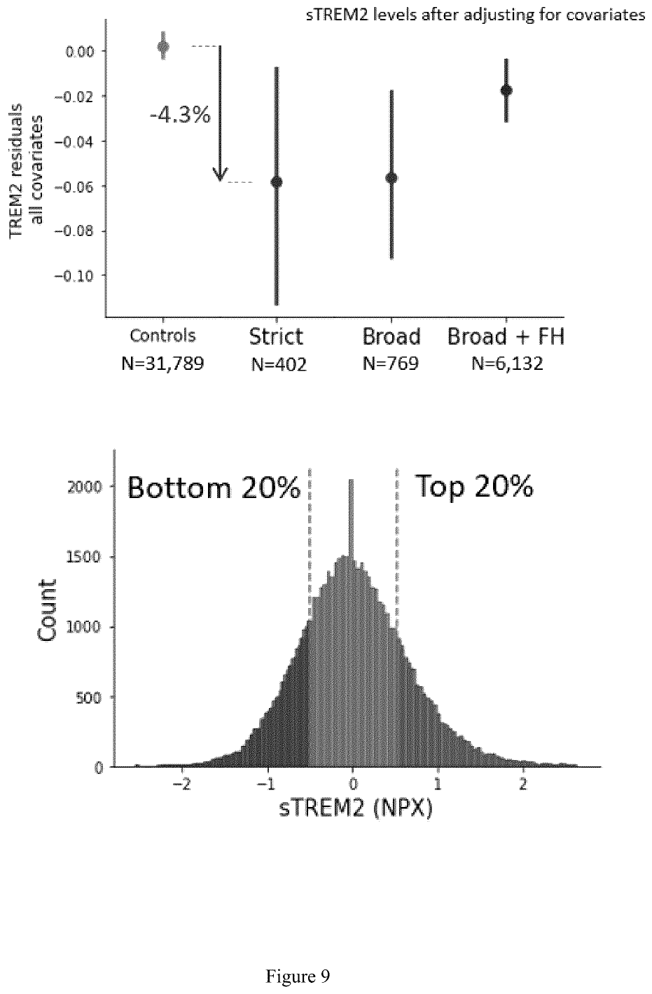

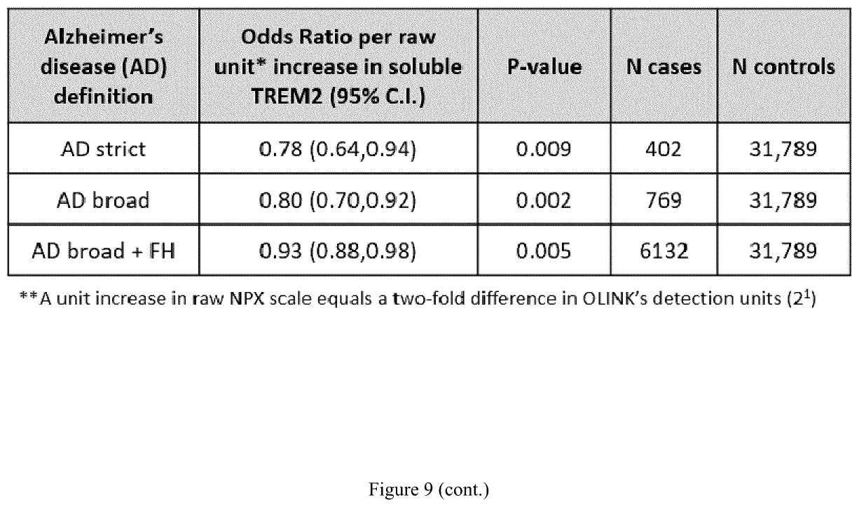

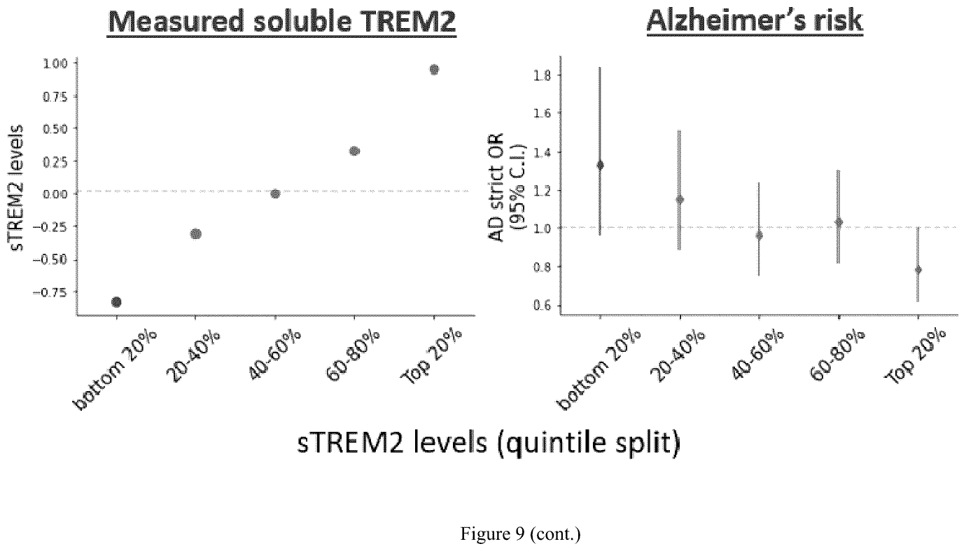

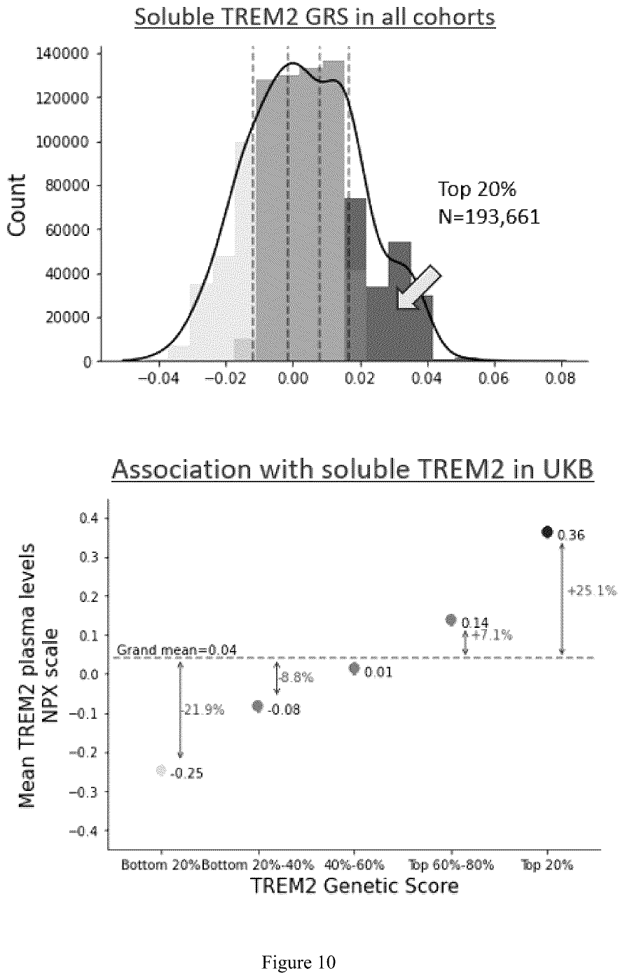

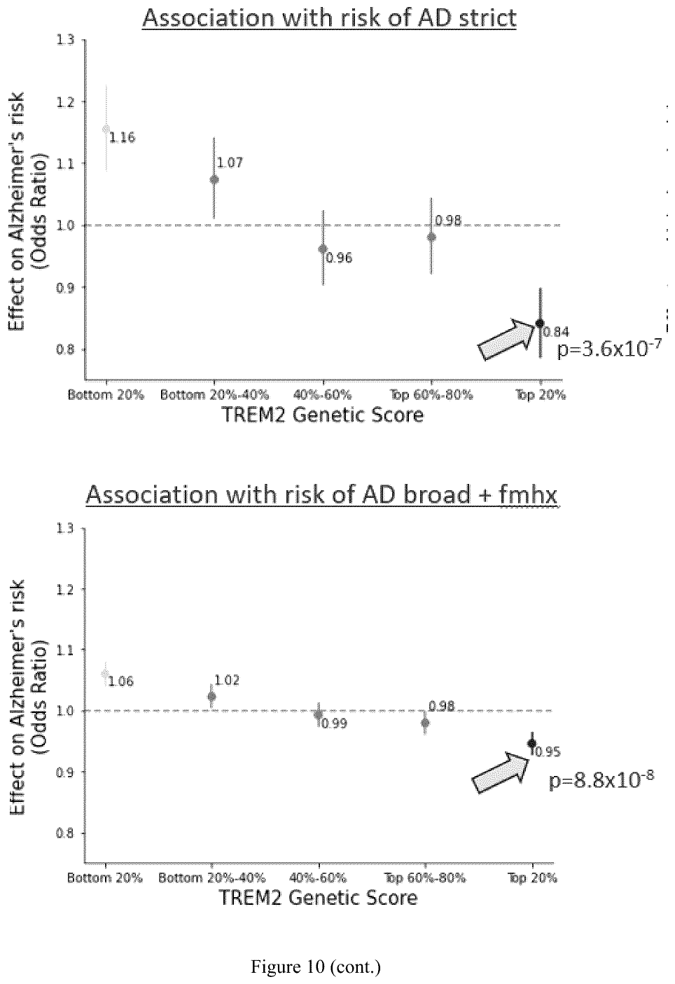

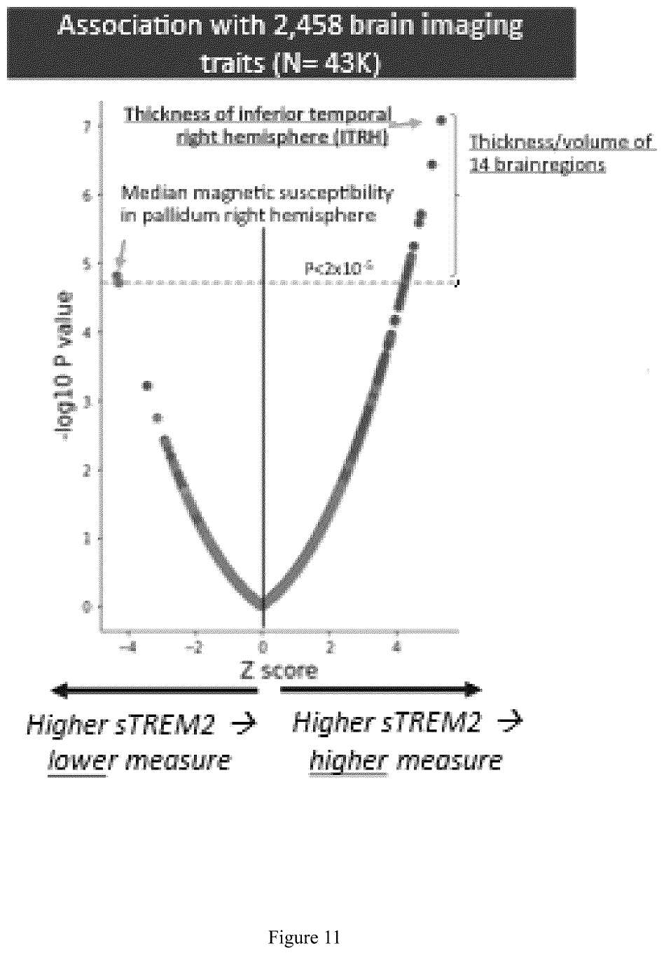

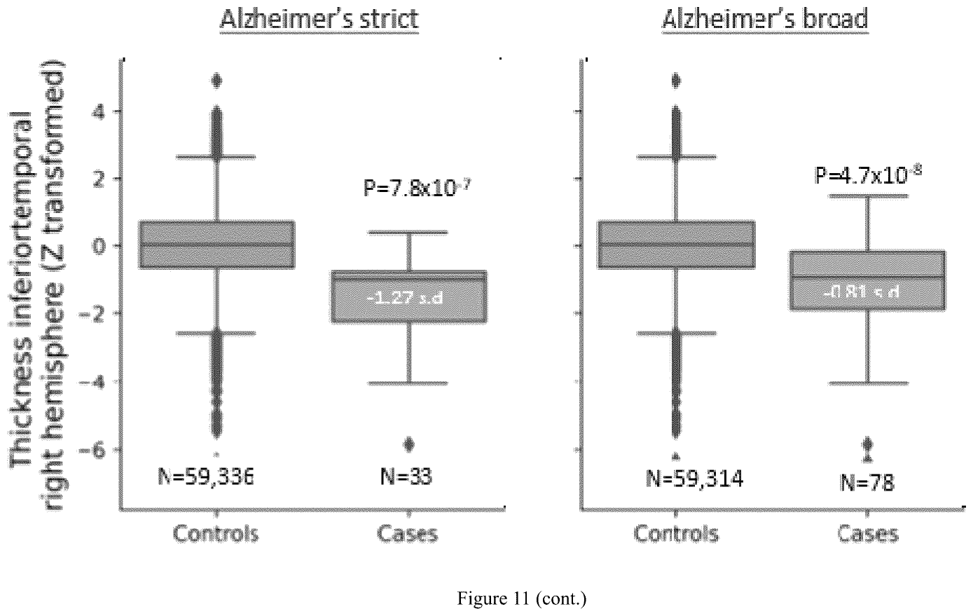

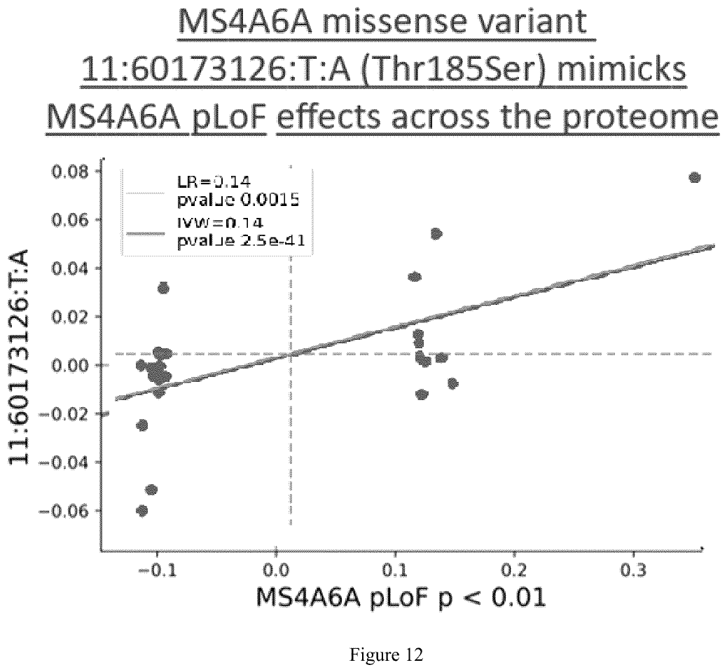

Six-month old female Ms4a6* triple KO mice received bilateral intrahippocampal injections of the two AAVs, 1E10 viral genomes/mouse for each virus. Three months post-injection, brains sections were analyzed via immunofluorescence using antibodies against Aβ (CST clone D54D2, #8243) and microglia (IBA1—Synaptic Systems #234 308). A trend was oibserved towards decreased Aβ deposit area in MS4A6* triple KO mice (FIG. 8), indicating that mice with decreased levels of a set of proteins with homology to human MS4A6A are protected from developing this hallmark AD pathology. Furthermore, Ms4a6* triple KO mice showed ˜40% decreased microglia clustering around Aβ deposits, defined as IBA1-positive area within 20 μm of Aβ deposits (FIG. 8). Taken together, these data suggest that in mice with decreased levels of proteins with homology to human MS4A6A, microglia function is altered, which may contribute to a trend towards decreased Aβ aggregate accumulation.

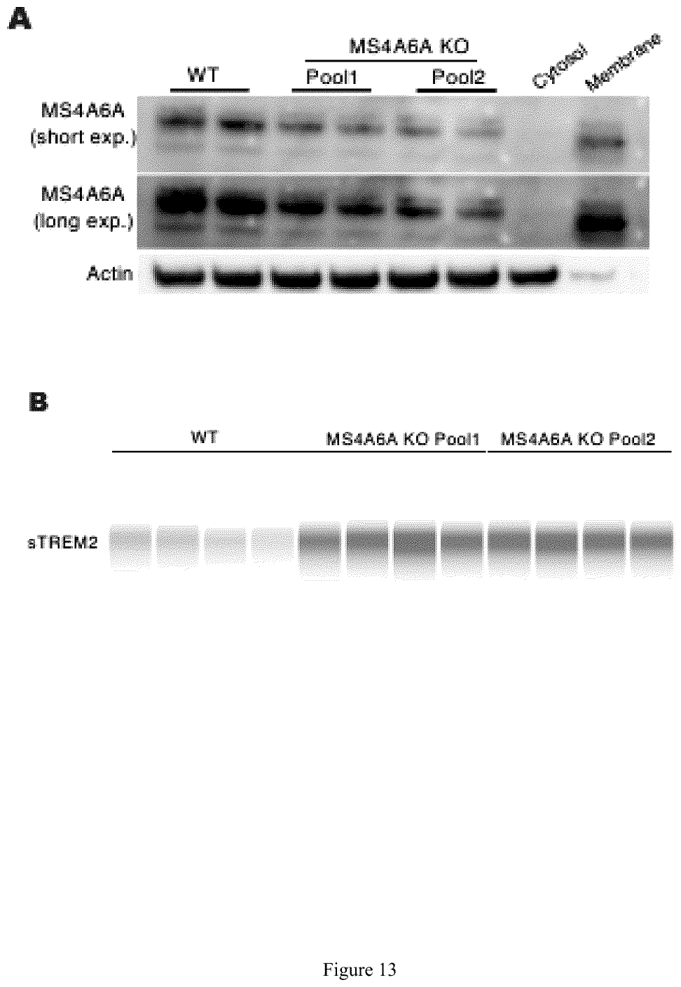

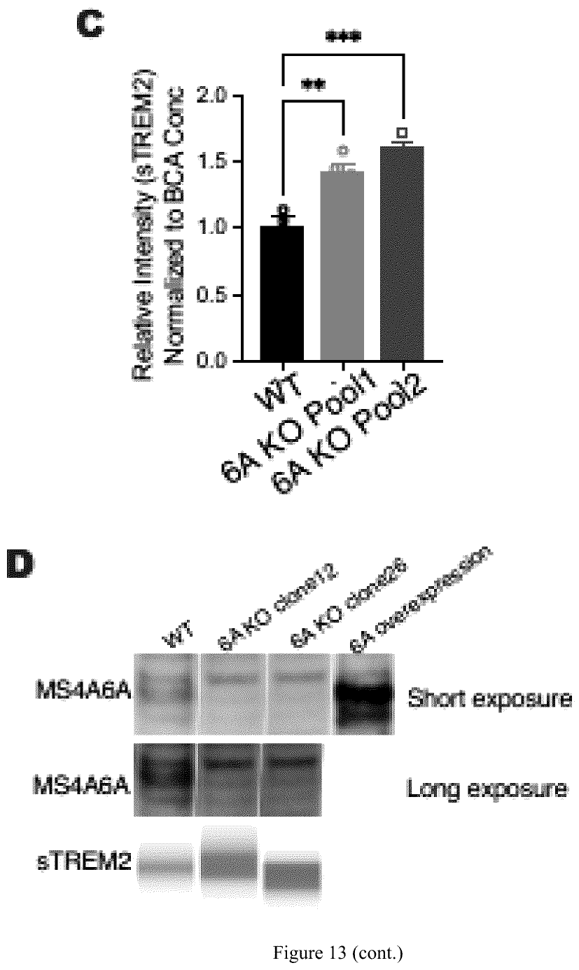

WT THP1 were transfected with Cas9-MS4A6A targeting gRNA complexes. After confirmation of successful genomic editing, cell lysates were prepared and analyzed by Western blot to confirm MS4A6A protein knockdown (see, FIG. 13, Panel A). Conditioned cell media were analyzed by JESS blot showing increased shed TREM2 in MS4A6A knockout pool compared to WT (see, FIG. 13, Panel B). Quantification of abundance was measured by JESS in Panel B (see, FIG. 13, Panel C). Representative MS4A6A WB and sTREM2 Jess in two different MS4A6A knockout THP1 lines were observed (see, FIG. 13, Panel D).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having myeloid cell dysfunction or at risk of developing myeloid cell dysfunction, the method comprising administering a Membrane Spanning 4-Domains A6A (MS4A6A) inhibitor or the combination of an MS4A6A inhibitor and a Membrane Spanning 4-Domains A4A (MS4A4A) inhibitor to the subject.

2. The method of claim 1, wherein the myeloid cell dysfunction is Alzheimer's disease (AD).

3. The method of claim 1, wherein the MS4A6A inhibitor and/or the MS4A4A inhibitor comprises an inhibitory nucleic acid molecule that hybridizes to an MS4A6A nucleic acid molecule or MS4A4A nucleic acid molecule.

4. The method of claim 3, wherein the inhibitory nucleic acid molecule comprises an antisense nucleic acid molecule, a small interfering RNA (siRNA), and/or a short hairpin RNA (shRNA).

5. The method of claim 4, wherein the inhibitory nucleic acid molecule comprises an siRNA.

6. The method of claim 4, wherein the inhibitory nucleic acid molecule comprises an antisense nucleic acid molecule.

7. The method of claim 1, wherein the subject is also administered a myeloid cell dysfunction agent.

8. The method of claim 1, further comprising detecting the presence or absence of an MS4A6A variant nucleic acid molecule in a biological sample from the subject.

9. The method of claim 8, further comprising administering a myeloid cell dysfunction agent in an amount that is the same as or less than a standard dosage amount to the subject when the MS4A6A variant nucleic acid molecule is absent from the biological sample.

10. The method of claim 8, further comprising administering a myeloid cell dysfunction agent in an amount that is the same as or less than a standard dosage amount to the subject when the subject is heterozygous for the MS4A6A variant nucleic acid molecule.

11. The method of claim 8, wherein the MS4A6A variant nucleic acid molecule comprises a splice-site variant, a stop-gain variant, a start-loss variant, a stop-loss variant, a frameshift variant, a missense variant, an in-frame indel variant, and/or a variant that encodes a truncated MS4A6A variant polypeptide.

12. The method of claim 8, wherein the MS4A6A variant nucleic acid molecule comprises any one or more of the genetic variations 11:60173027:C:T (Val218Met, Val246Met), 11:60179911:AT:A, 11:60173126:T:A (Thr185Ser), or 11:60181585:A:G (Ile76Thr), 11:60173027:C:T (splice donor), or 11:60179911:AT:A (pSer68fs).

13. A method of treating a subject having myeloid cell dysfunction or at risk of developing myeloid cell dysfunction by administering a myeloid cell dysfunction agent, the method comprising:
determining or having determined whether the subject has a Membrane Spanning 4-Domains A6A (MS4A6A) variant nucleic acid molecule, by:
obtaining or having obtained a biological sample from the subject; and
performing or having performed a sequence analysis on the biological sample to determine if the subject has a genotype comprising an MS4A6A variant nucleic acid molecule; and
administering or continuing to administer the myeloid cell dysfunction agent in an amount that is the same as or less than a standard dosage amount, and/or an MS4A6A inhibitor or the combination of an MS4A6A inhibitor and an MS4A4A inhibitor to a subject that is MS4A6A reference;
administering or continuing to administer the myeloid cell dysfunction agent in an amount that is the same as or less than a standard dosage amount, and/or an MS4A6A inhibitor or the combination of an MS4A6A inhibitor and an MS4A4A inhibitor to a subject that is heterozygous for the MS4A6A variant nucleic acid molecule; or
administering or continuing to administer the myeloid cell dysfunction agent in a standard dosage amount to a subject that is homozygous for the MS4A6A variant nucleic acid molecule;
wherein the presence of the MS4A6A variant nucleic acid molecule indicates the subject has a decreased risk of developing myeloid cell dysfunction.

14. The method of claim 13, wherein the MS4A6A inhibitor and/or MS4A4A inhibitor comprises an inhibitory nucleic acid molecule that hybridizes to an MS4A6A nucleic acid molecule or an MS4A4A nucleic acid molecule.

15. The method of claim 14, wherein the inhibitory nucleic acid molecule comprises an antisense nucleic acid molecule, a small interfering RNA (siRNA), and/or a short hairpin RNA (shRNA).

16. The method of claim 15, wherein the inhibitory nucleic acid molecule comprises an siRNA.

17. The method of claim 15, wherein the inhibitory nucleic acid molecule comprises an antisense nucleic acid molecule.

18. The method of claim 13, wherein the subject is heterozygous for the MS4A6A variant nucleic acid molecule, and the subject is administered or continued to be administered the myeloid cell dysfunction agent in an amount that is the same as or less than a standard dosage amount and the MS4A6A inhibitor or the combination of the MS4A6A inhibitor and the MS4A4A inhibitor.

19. The method of claim 13, wherein the subject is MS4A6A reference, and the subject is administered or continued to be administered the myeloid cell dysfunction agent in an amount that is the same as or less than a standard dosage amount and the MS4A6A inhibitor or the combination of the MS4A6A inhibitor and the MS4A4A inhibitor.

20. The method of claim 13, wherein the MS4A6A variant nucleic acid molecule comprises a splice-site variant, a stop-gain variant, a start-loss variant, a stop-loss variant, a frameshift variant, a missense variant, an in-frame indel variant, and/or a variant that encodes a truncated MS4A6A variant polypeptide.

21. The method of claim 13, wherein the MS4A6A variant nucleic acid molecule comprises any one or more of the genetic variations 11:60173027:C:T (Val218Met, Val246Met), 11:60179911:AT:A, 11:60173126:T: A (Thr185Ser), or 11:60181585:A:G (Ile76Thr), 11:60173027:C:T (splice donor), or 11:60179911:AT: A (pSer68fs).

22. A method of identifying a subject having an increased risk of developing myeloid cell dysfunction, the method comprising:
determining or having determined the presence or absence of a Membrane Spanning 4-Domains A6A (MS4A6A) variant nucleic acid molecule in a biological sample obtained from the subject;
wherein:
when the subject is MS4A6A reference, then the subject has an increased risk of developing myeloid cell dysfunction; and
when the subject is heterozygous or homozygous for the MS4A6A variant nucleic acid molecule, then the subject has a decreased risk of developing myeloid cell dysfunction.

23-49. (canceled)
